(19) United States
(12) Patent Application Publication
(10) Pub. No.: US 2002/0151489 A1
(43) Pub. Date: Oct. 17, 2002

(54) Use of lymphangiogenic agents to treat lymphatic disorders

(75) Inventors: Edwin C. Gravereaux, Brookline, MA (US); Marcy Silver, Bolton, MA (US); Young-Sup Yoon, Watertown, MA (US); Jeffrey M. Isner, Weston, MA (US); Linda Isner, Weston, MA (US)

Correspondence Address: David G. Conlin, Dike, Bronstein, Roberts & Cushman, Intellectual Property Practice Group, P. O. Box 9169, Boston, MA, 02209, US

(73) Assignee: St. Elizabeth's Medical Center of Boston, Inc.

(21) Appl. No.: 09/970,088

(22) Filed: Oct. 2, 2001

Related U.S. Application Data
(60) Provisional application No. 60/237,171, filed on Oct. 2, 2000.

(51) Int. Cl. A61K 38/18

(52) U.S. Cl. 514/12

(57) ABSTRACT

The present invention provides methods for promoting the growth of new lymph vessels (lymphangiogenesis). Generally, such methods include administering at least one vascular endothelian factor (VEGF) such as VEGF-2. In one embodiment, therapeutic methods for treating lymphedema and related disorders in a human patient are disclosed. The VEGF can be provided by any suitable means including direct injection of a nucleic acid encoding same or an active fragment thereof. Also provided are pharmaceutical products for promoting lymphangiogenesis as well as a test system for screening compounds capable of inducing new lymph vessel growth.

### CROSS-REFERENCE TO RELATED APPLICATION

The present application is a continuation-in-part of U.S Provisional Application No. 60/237,171 filed on Oct. 2, 2000. The disclosure of said provisional application is incorporated herein by reference.

### FIELD OF THE INVENTION

The present invention generally relates to compositions and methods for modulating lymph vessel growth in a mammal. In one aspect, methods are provided for modulating new lymph vessel growth (lymphangiogenesis) that include administrating an effective amount of at least one vascular endothelian growth factor (VEGF) such as VEGF-2. Further provided are methods for treating lymphedema and other conditions impacting the lymphatic system. Also provided is a test system for screening compounds capable of inducing lymphangiogenesis. The invention has a wide spectrum of useful applications including promoting good lymphatic function in the mammal.

### BACKGROUND

There is recognition that lymphedema is a progressive, usually painless extremity swelling secondary to decreased transport capacity of the lymphatic system. The condition, despite being painless, can cause extreme distress and functional impairment, leading to frequent disability in those afflicted. In addition to the often massive size of the affected limb, secondary cellulitis and lymphangitis episodes complicate the condition. The late risk of lymphangiosarcoma arising in the lymphedematous limb remains a concern.

Significantly, the exact pathogenesis of lymphedema remains uncertain. However, the cause of lymphedema can usually be divided into primary or secondary groupings.

The former (primary group) includes developmental abnormalities of the lymphatic system (aplasia, hypoplasia, or hyperplasia with valvular incompetence), and the most frequent causes of acquired lymphedema remain iatrogenic (post-surgical, traumatic), infectious, or related to tumor invasion.

There is general understanding about the structure and function of the lymphatic system. For example, the skin lymphatic system consists of the initial lymphatics, which converge into lymphatic precollectors, collectors, and lymphatic ducts. These in turn convey the lymph to the regional lymph nodes. Interstitial fluid and particles enter the initial lymphatics through inter endothelial openings and by vesicular transport. Lymphatic uptake is enhanced by external compression, but also depends greatly upon contraction of lymphangions which generate the suction force promoting absorption of interstitial fluid and expulsion of lymph to the collecting ducts.

In lymphedema, various types of congenital and acquired abnormalities of lymphatic vessels and lymph nodes have been observed. These often lead to lymphatic hypertension, valvular insufficiency, and lymphostasis. Accumulation of interstitial and lymphatic fluid within the skin and subcutaneous tissue stimulates fibroblasts, keratinocytes, and adipocytes leading to deposition of collagen and glycosaminoglycans, together with skin hypertrophy and destruction of elastic fibers.

There has been efforts to understand and classify primary lymphedema.

Three types of primary lymphedema are thought to exist: 1) congenital, present at birth or within two years of life; 2) precox, the most common subtype, occurring at puberty or by the third decade of life; and 3) tarda, with onset after 35 years of age.

In particular, congenital lymphedema may have a familial distribution, with an autosomal dominant pattern of transmission described (Milroy disease), however, sporadic cases are more common. Swelling usually involves only one lower extremity, but involvement can include multiple limbs, genitalia or the face. A higher proportion of males are affected. See e.g., Witte, M. H et al. (1998) in Lymphology 31: 145.

Lymphedema precox is the most frequent form of primary lymphedema. Meige disease is reserved for specific familial forms with a recessive inheritance pattern. Precox is much more common in females, with a 10:1 female to male ratio. Edema is usually unilateral and limited to the foot and calf in most patients. There is belief that estrogens may be involved in the pathogenesis of the disease state as onset often coincides with puberty.

Lymphedema tarda occurs after age 35. Approximately 10% of congenital lymphedema cases fall into this grouping.

A functional classification of primary lymphedema has been proposed based upon underlying lymphatic anatomy demonstrated by lymphangiography, with three different anatomical abnormalities seen, each associated with different clinical presentations. This classification scheme, importantly, centers around selection of groups which may be responsive to medical or surgical therapies.

1.

Distal Obliteration. Distal obliteration on lymphangiography comprises 80% of patients, predominantly female, and with bilateral involvement. There are decreased or absent superficial leg Lymphatics (aplasia or hypoplasia). Progression of edema is slow and is often responsive to compression therapy.

2.Proximal Occlusion.

Proximal occlusion of aorto-iliac or inguinal Lymph nodes occurs in 110%to of primary lymphedema cases. This picture is usually unilateral with edema usually involving the entire lower extremity. The edema can develop rapidly and responds poorly to conservative treatment. If associated with distal lymphatic dilatation, mesenteric budge surgery or microvascular Lymphatic reconstruction may be helpful.

3.Hyperplasia.

Hyperplasia with incompetence of Lymphatics is seen in the remaining 10% of patients. Bilateral edema is present. A subgroup has megalymphatics, and chylous reflux can result from concomitant involvement of mesenteric Lymphatic reflux. Chylous drainage from small vesicles can be seen in the genitalia and lower extremities, and these patients are candidates for surgical ligation and excision of incompetent retroperitoneal lymphatics.

There have been efforts to understand secondary lymphedema.

In particular, secondary lymphedema develops as a consequence of disruption or obstruction of Lymphatic pathways by surgery or other disease processes, and is considerably more common than is the primary form.

1. Iatrogenic Lymphedema

Disruption of lymphatic pathways can be caused by surgery and/or radiation therapy, which produces fibrosis. These may be intentional or accidental, with the most common modem examples being arm edema in women after mastectomy with axillary node dissection for breast cancer and leg edema after inguinal and pelvic lymph node dissection for pelvic neoplasms. Incidences of post mastectomy edema vary widely among published series, from ˜80%, with extent of surgery, subsequent radiation use, and obesity correlating with development of edema. Its prevalence may be underestimated as milder degrees of lymphedema can easily be overlooked. Interestingly, lymphatic-venous communications have been documented by lymphoscinitigraphy, and one proposal is that open lympho-venous channels serve as safety valves for overloaded lymphatics and could prevent edema. With lymphoscintigraphy, it has been possible to demonstrate the presence of lymph-venous anastamoses in non-edematous post mastectomy patients, while those with lymphedema lack evidence of lymphovenous communications. Edema of the leg is comparably common after pelvic surgery, especially with the addition of lymph node dissection and radiation. Lymphedema has also been seen after vascular procedures involving the iliac and femoral vessels, especially with repeat surgeries.

2. Post-Infectious Lymphedema

Lymphedema can occur after severe single or repeated bouts of streptococcal cellulitis or lymphangitis with resultant swelling of the limb. This inflammatory edema has decreased in incidence, likely attributable to widespread antibiotic use. Filariasis is the most common cause of lymphedema worldwide with up to 90 million people estimated to be infected. Most symptomatic patients have lymphedema and endemic areas large percentages of the population can be affected. Pathologic mechanisms for edema development in these patients include direct toxic effect of the worms, the resultant immune response, and superimposed bacterial infection.

3. Neoplastic Disease and Other Etiologies

Neoplastic obliteration of lymphatic lymph node metastases, and external compression by tumor are major causes of secondary lymphedema. Other isolated causes include lymphedema accompanying rheumatoid and psoriatic arthritis and lymphedema can be seen with other types of chronic edema, such as chronic venous insufficiency and lipedema.

See FIGS. 1A-C (showing various lymphedema classifications).

There have been attempts to diagnose lympedema as follows.

In most instances, a typical history and characteristic clinical picture are sufficient to establish the diagnosis of lymphedema. But, diagnosis may be difficult early or when the edema is mild or intermittent. additional tests can confirm the presence of impaired lymphatic flow and/or the typical pattern of abnormal fluid distribution in the tissues.

Lymphoscintigraphy

Using radiolabeled macromolecular tracer (99 Tc-sulfur colloid), intra or subdermal injection allows tracking of Lymphatic transport using a gamma camera. The rate of tracer disappearance from the injection site and accumulation of counts within Lymph node basins are quantifiable. Typical abnormalities seen in lymphedema include dermal backflow, absent or delayed transport of tracer or absent or delayed lymph node visualization. This remains the best of the readily available methods to evaluate lymphatic function.

Magnetic Resonance Imaging

Reveals distribution in lymphedema of edema within epifascial compartment, with honeycombing of the subcutaneous tissue and skin thickening. In venous edema, both epi and subfascial compartments are affected, and in lipedema, fat accumulates without fluid. MRI can also aid in anatomic identification of lymph nodes, enlarged lymphatic trunks, and help in differentiating various causes of Lymphatic obstruction in secondary lymphedema. New contrast media may have promising applications.

CT Scan

Provides anatomic definition of edema location (sub vs epifascial) and can identify skin thickening and honeycombing of subcutaneous tissue in lymphedema. CT may have a role m monitoring responses to therapy through serial measurements of cross-sectional area and tissue density.

Indirect Lymphangiography

Utilizes water soluble iodinated contrast media that are infused intradermally and enter the lymphatics. Visualization of lymphatics is obtained using xray, and can be specifically useful to visualize skin lymphatics and Lymphatic trunks, which may be helpful prior to reconstructive surgery attempts.

Ultrasound

Is utilized as a complementary tool for the noninvasive evaluation of the swollen extremity. In lymphedema, thickening of the cutaneous and epifascial compartments has been observed, and may aid in diagnosis and therapeutic monitoring.

There have been reports of complications associated with lymphedema.

For example, lymphangitis/cellulitis can often complicate longstanding lymphedema, with the accumulated proteins in the edema fluid serving as culture media for bacterial growth. A vicious circle of bacterial proliferation, secondary to impaired immune response due to impaired lymphatic drainage, further damages remaining lymphatic capillaries and aggravates the edema. Prophylaxis includes meticulous skin care, avoidance of trauma, and edema reducing treatment, with or without prophylactic antibiotics.

In addition, malignant tumors can infrequently arise in the edematous limb, most often observed in the arm after mastectomy following a long latency period. The lymphangiosarcoma is very aggressive with low survival rates. Other cancers observed include Kaposi's sarcoma, squamous cell carcinoma, malignant lymphoma, and melanoma.

There have been proposals to treat lymphedema as follows.

Most treatment methods include mechanical reduction of the swollen limb by elevation, massage, pneumatic compression therapy, and heat therapy. Graduated elastic support stockings are used to attempt to maintain the limb size. There is presently no cure for lymphedema, and only restoration of lymph-transporting capacity can be imagined to deal specifically with the cause of the lymphedema, that is, the insufficient lymphatic drainage of the limb. Surgical attempts at reconstructing the obstructed lymphatic pathways include lymphovenous anastomoses, lymphatic grafting, and autotransplantation of lymphatic tissue. None have shown consistent or reproducible long-term effectiveness.

There have been reports that vascular endothelial factor 2 (VEGF-2, sometimes called VEGF-C) can assist lymphatic hyperplasia and angiogenesis in some settings. See Jeltsch, M. et al. (1997) Science 276: 1423; and Oh, S. J. et al. (1997) Dev. Biol. 188: 96. However it is unclear whether such activity can be used to treat lymphedema, particularly in a patient.

Accordingly, there is an urgent need for methods of treating lymphedema. More particularly, there is a need for new therapies that can help grow neo-lymphatic vessels in patients. There is also a need for reliable animal models that can be used to test compounds for lymphangiogenic activity.

### SUMMARY OF THE INVENTION

The present invention generally relates to methods for modulating lymph vessel growth in a mammal. In one aspect, the invention provides methods for increasing new lymph vessel growth that include administrating an effective amount of a vascular endothelian factor (VEGF) such as VEGF-2 or an effective fragment thereof. The invention also relates to methods for treating lymphedema and related disorders in the mammal. The invention has many uses including preventing or reducing the severity of lymphedema in human patients.

We have now discovered that VEGF and especially VEGF-2 modulates growth of new blood vessels in human patients. In particular, we have found that VEGF-2 promotes growth of new lymph vessels in response to lymphedema. This observation was surprising and unexpected in light of prior reports addressing VEGF-2 activity in vitro and in vivo. Accordingly, this invention provides methods for using VEGFs such as VEGF-2 as well as isoforms, allelic variants and effective fragments thereof to promote lymphangiogenesis especially in tissues in need of such new vessels.

Accordingly, and in one aspect, the invention features a method for inducing formation of new lymphatic vessels in a mammal e.g., a rodent, rabbit or primate. Preferably, the method includes administering to the mammal an effective amount of VEGF, preferably VEGF-2 or an isoform, allelic variant, mutein or effective fragment thereof sufficient to form the new vessels in the mammal.

In a preferred example of the method, the amount of the VEGF administered to the mammal is sufficient to decrease ear volume by at least about 10% as determined by a standard rabbit ear assay. It is also preferred that the amount of the VEGF administered to the mammal is sufficient to increase the number of lymphatic vessels by at least about 10% as determined by a standard lymphoscintigraphy assay. Preferably, the VEGF so administered is VEGF-2 including VEGF-2 muteins; or active fragments thereof. The standard rabbit ear and lymphoscintigraphy assays are discussed below.

As will be apparent, the invention is useful for reducing the severity of lymphedema and other conditions impacted by aberrant lymphatic function including lymphangietasia, lymphangioma, and lymphangiosarcoma. The lymphedema may be of the primary or secondary type as shown in the Drawings. See e.g., FIGS. 1A-C and 2.

In some instances, it may be desirable to enhance angiogenesis before, during or after support of new lymph vessel growth. For example, severe limb trauma may require the growth of new lymph vessels and blood vessels. This can be achieved by one or a combination of different strategies including administering at least one angiogenic protein to induce new blood vessel growth and at least one lymphangiogenic protein to encourage growth of new lymph vessels. Routes involving co-administration of the angiogenic protein with at least one lymphangiogenic protein are generally preferred.

By the term “induction” is meant at least enhancing lymphangiogenesis and optionally angiogenesis as well. More specifically, the word is meant to denote formation of lymph vessels and optionally formation of blood vasculature in the mammal.

The invention also encompasses a method for preventing or reducing the severity of lymphatic vessel damage in a mammal. In one embodiment, the method includes administering to the mammal an effective amount of vascular endothelian growth factor (VEGF), such as VEGF-2 including isoforms, allelic variants, muteins and active fragments thereof; and exposing the mammal to conditions conducive to damaging the lymphatic vessels, the amount of VEGF being sufficient to prevent or reduce the severity of the vessel damage in the mammal.

The invention also features methods for treating lymphedema in a mammal in need of such treatment. In one example, the method includes administering to the mammal an effective amount of vascular endothelial growth factor (VEGF), VEGF-2; or a mutein, isoform, allelic variant or effective fragment thereof sufficient to form the new vessels in the mammal.

Also provided by the present invention are methods for treating lymphedema in a mammal in need of such treatment. In one embodiment, the methods include administering to the mammal an effective amount of vascular endothelial growth factor (VEGF), VEGF-2; an isoform, allelic variant mutein or effective fragment thereof sufficient to form the new vessels in the mammal. In another embodiment, the invention further includes co-administering at least one angiogenic protein to the mammal.

The invention also features a pharmaceutical product for inducing growth of new lymphatic vessels in a mammal. In one embodiment, the product comprises vascular endothelian factor 2 (VEGF-2) including isoforms, allelic variants, muteins and effective fragments thereof. More preferred products are formulated to be physiologically acceptable to a mammal. The pharmaceutical product is typically provided sterile and will include e.g., VEGF-2 protein or nucleic acid encoding the protein.

Also within the scope of this invention is a kit for the treating lymphedema in a human patient. In an example, the kit includes, e.g., VEGF-2 protein, nucleic acid encoding VEGF-2, or an effective fragment thereof. Optionally included in the kit is a pharmacologically acceptable carrier solution, means for delivering the VEGF-2 protein or nucleic acid and directions for using the kit.

In another aspect, the invention features a test system for identifying compounds that reduce lymphedema. In one embodiment the system includes:

a) a mammal characterized by having a surgically manipulated appendage such as an ear or limb, the manipulation being sufficient to expose a neurovascular bundle (NVB) in the appendage and to provide a substrate for detecting neolymphatic growth, preferably the mammal is a rabbit or other large-eared herbivore.

b) a candidate compound for reducing lymphedema in the mammal such as VEGF such as VEGF-2 as well as isoforms, allelic variants, muteins and effective fragments thereof; and

c) at least one implementation or assay e.g., calipers or water volume assay, for detecting an increase or decrease in appendage thickness following contact of the candidate compound with the NVB.

The invention will be more fully appreciated by reference to the following drawings.

### BRIEF DESCRIPTION OF THE DRAWINGS

FIGS. 1A-C are tables showing various reported lymphedema classifications FIG. 2 is a drawing showing lymphangiographic patterns in normal patients and primary lymphedema.

FIG. 3 is a photograph illustrating a rabbit ear lymphedema model. The photographs show clinical appearance after five (5) months.

FIG. 4 is a photograph showing lymphoscintigraphy of the rabbit ear lymphedema model five (5) months post-op.

FIG. 5 is a photograph showing lymphoscintigraphy orientation in the rabbit ear lymphedema model.

FIG. 6 is a photograph illustrating lymphoscintigrapy-early post op in the rabbit ear lymphedema model.

FIG. 7 is a graph showing ear volume versus days post-administration of VEGF.

FIG. 8 is a photograph showing severe lymphedema 3 days post-op in the rabbit ear lymphedema model.

FIG. 9 is a photograph showing results of human lymphoscintigraphy. The photograph shows that direct gene transfer of VEGF-2 DNA promotes new lymphatic channels (post-VEGF-2) that were not present in the control (pre-VEGF-2).

FIG. 10 is a photograph showing ultrasound imaging of intramuscular VEGF-2 gene transfer: lymphedema.

FIG. 11 is a photograph showing antibody staining for lymphatic vessels in a patient.

FIGS. 12A-C are pictures showing the rabbit ear lymphedema model.

FIGS. 13A-B exemplify gene transfer into the rabbit ear lymphedema model. FIG. 13A shows a picture of the model. FIGS. 13B is a drawing showing a preferred gene transfer protocol.

FIGS. 14A-C are drawings showing an example of lymphoscintigraphy.

FIGS. 15A-C show radioactive quantification using the rabbit ear lymphedema model. FIG. 15A-B are lymphoscintigrams. FIG. 15C is a graph summarizing results.

FIGS. 16A-B are graphs showing ear thickness and volume.

FIGS. 17A-D are pictures further exemplifying the rabbit ear lymphedema model. FIGS. 17A-B show rabbit ear pictures. FIGS. 17C-D show lymphoscintigrams.

FIGS. 18A-B show results of microscopic examination of rabbit ears. FIG. 18A are pictures of tissue sections. FIG. 18B is a graph summarizing results. FIGS. 19A-J are pictures showing results of lymphoscintigraphic findings.

FIGS. 20A-B show transgene expression of VEGF-C in various tissues.

FIG. 21 is a drawing showing a partial sequence of the rabbit VEGFR-3 cDNA sequence. Also shown, for comparison, are bovine, human and mouse sequences.

FIG. 22A is a drawing showing the amino acid sequence encoded by the rabbit nucleic acid sequence of FIG. 21. FIGS. 22B-C show results of RT-PCT experiments. Results of those experiments are summarized in FIG. 22D.

FIGS. 23A-B show results of VEGF-C transgene expression in the mouse tail model. FIG. 23C is a graph summarizing results.

FIGS. 24A-C shows results of antibody staining of the LYVE-1 lymphatic vessel antigen. FIG. 24D summarizes results in a graph.

### DETAILED DESCRIPTION OF PREFERRED EMBODIMENTS

As discussed, the present invention provides, in one aspect, methods for inducing the growth of new lymph vessels (lymphangiogenesis) particularly in a human patient that include administrating to the patient an effective amount of VEGF, preferably VEGF-2 as well as effective muteins, isoforms, allelic variants and fragments thereof. As also discussed, that VEGF-2 can be administered to the human patient alone or in combination (co-administered) with an angiogenic protein particularly in settings in which good growth of lymph and blood vessels are desired. Also provided are pharmaceutical compositions for promoting lymphangiogenesis. The invention has a wide spectrum of uses including preventing or reducing the severity of lymphedema in a human patient.

By the term “VEGF” is meant one of the family of vascular endothelian growth factors related to PIGF. Preferred members include VEGF, VEGF-1 (VEGFA), VEGF-2 (VEGFC), VEGF-3 (VEGFB). The term is also meant to include isoforms, muteins, allelic variants, and effective fragments thereof showing good (at least about 10%) activity in the rabbit ear assay.

See generally Olofsson, B. et al. Current Opinion in Biotechnology 10: 528 (1999) and references cited therein, the disclosure of which is incorporated by reference.

The invention particularly provides methods for inducing lymphangiogenesis in patients in need of such treatment such as those having or suspected of having lymphedema. In this embodiment, the methods generally include administering to the patient an effective amount of VEGF-2 or other suitable protein disclosed herein. Administration of the VEGF-2 (or coadministration with other another protein or proteins) can be as needed and may be implemented prior to, during or after formation of the ischemic tissue. Additionally, the VEGF-2 can be administered as the sole active compound or it can be co-administered with at least one and preferably one angiogenic protein or other suitable protein or fragment as provided herein.

Administration of an effective amount VEGF-2 or other protein disclosed herein in accord with any of the methods disclosed herein can be implemented by one or a combination of different strategies including administering a DNA or RNA encoding same.

As discussed, methods of this invention have a wide spectrum of uses especially in a human patient, e.g., use in the prevention or treatment of at least one of lymphedema as well as other disorders referred to herein. Impacted tissue can be associated with nearly any physiological system in the patient including the circulatory system or the central nervous system, e.g., a limb, graft (e.g., muscle or nerve graft), or organ (e.g., heart, brain, kidney and lung).

In embodiments in which an effective amount of the VEGF-2 or other suitable protein is administered to a mammal and especially a human patient to prevent or reduce the severity of a vascular condition and particularly ischemia, the VEGF-2 will preferably be administered at least about 12 hours, preferably between from about 24 hours to 1 week up to about 10 days prior to exposure to conditions conducive to damaging blood vessels. If desired, the method can further include administering the VEGF-2 to the mammal following exposure to the conditions conducive to damaging the blood vessels.

Good lymphangiogeneis can be monitored if desired by a combination of standard routes including lymphoscintigraphy and related approaches. A standard lymphoscintigraphy assay is provided below.

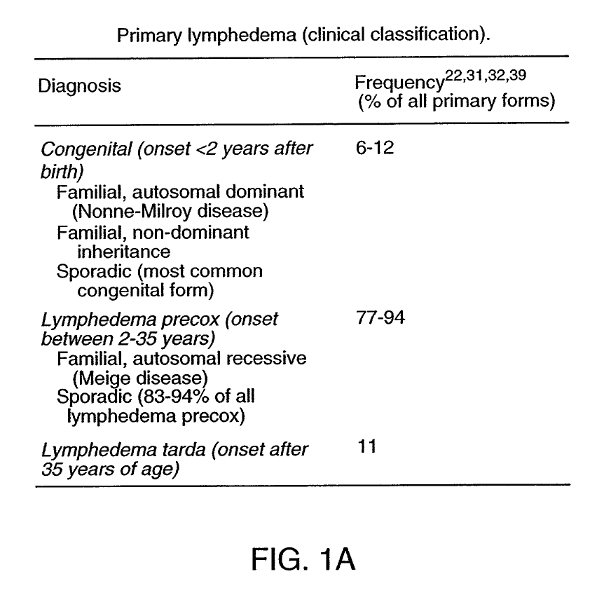

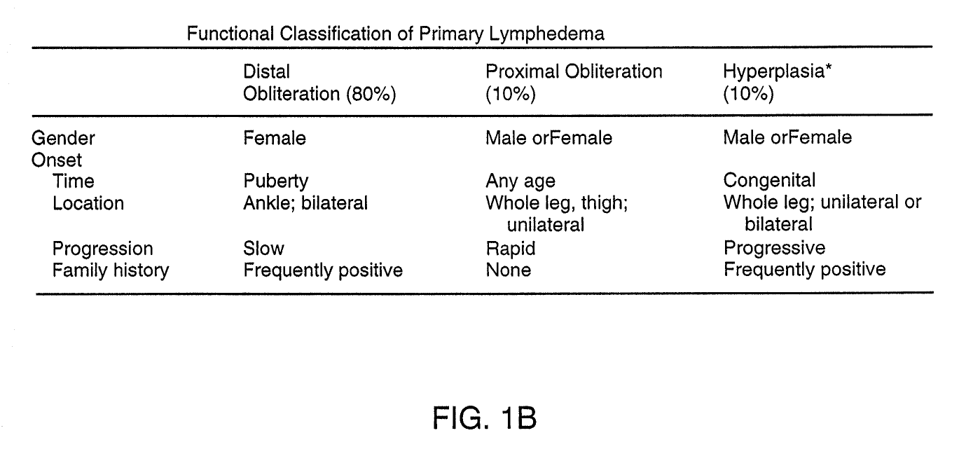

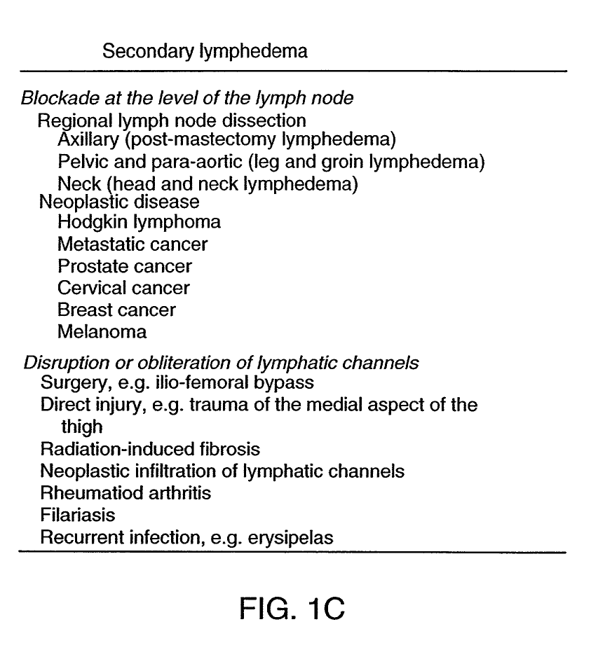

Lymph vessel injury is known to be facilitated by one or a combination of different tissue insults. For example, such injury often results from tissue trauma, surgery, cancer, genetic disorders as well as other medical conditions disclosed herein. For a summary, see FIGS. 1A-1C.

As discussed above and in the Examples following, we have discovered means to promote lymphangiogenesis in mammals. These methods involve the use of VEGF-2 to mobilize lymph vessel precursor cells. In accordance with the present invention, VEGF-2 can be used in a method for enhancing lymphangiogenesis in a selected patient having lymphedema i.e., an extremity or tissue having a deficiency in lymph vasculature and related lymph drainage as a direct or indirect result of recognized conditions. See FIGS. 1A-1C.

FIG. 2 provides a specific illustration of the kinds of lymphedema problems found in many human patients. In most cases, vessel obstruction is a major problem. Accordingly, the growth of new lymphatic channels in accord with this invention would address about 92% of the patients who present lymphedema due to obliterated lymphatics as opposed to the smaller hyperplastic group.

A spectrum of conditions are known to impact lymphedema. Many of these are summarized in FIGS. 1A-1C.

For example, conditions conducive to lymphatic vessel damage include an invasive manipulation, disease, genetic predisposition, congential (onset less than about two years after birth), lymphedema precox, lymphedema tarda, or trauma such as that associated with a medial aspect of the thigh. In one example, the invasive manipulation is surgery such as ilio-femoral bypass, regional lymph node dissection including axillary (post-mastectomy lymphedema), pelvic and para-aortic (leg and groin lymphedema), and neck (head and neck lymphedema). In another example, the disease is a neoplastic disease, rheumatoid arthritis, Filariasis or recurrent infection such as erysipelas. In particular, the neoplastic disease can be hodgkin lymphoma, metastatic cancer, or a cancer of the prostate or breast, cervical cancer or melanoma.

Preferred examples of genetic pre-disposition to lymphedema include a familial autosomal dominant pre-disposition such as Nonne-Milroy disease. However other examples are familial and non-dominant. Also, congential lymphatic vessel damage can, in some circumstances, be sporadic.

Examples of lymphedema precox (onset between about 2 and 35 years of age) include familial, and autosomal recessive such as Meige disease. Sporadic type is also known.

In some invention embodiments, the lymphedema may be primary or secondary. In situations in which primary lymphedma is an issue it can be associated with one or more of a distal obliteration, proximal obliteration, or hyperplasia.

By the term “lymphangiogenic agent” or “lymphangiogenic protein” is meant any protein, polypeptide, mutein, or portion thereof capable of, directly or indirectly, inducing the formation of new lymph vessels. A preferred lymphangiogenic protein is more particularly capable of reducing ear volume by at least about 10%, preferably about 20% to about 40%, more preferably at least about 50% to about 70%, as determined in the standard rabbit ear assay described herein. An example of such a protein or agent is vascular endothelian factor (VEGF), particularly VEGF-2 including fragments and muteins thereof showing activity in the rabbit ear assay.

Reference herein to a “standard rabbit ear assay” or similar phrase means an assay that includes at least one and preferably all of the following steps:

a) surgically manipulating at least one ear of a mammal such as a rabbit to expose a neurovascular bundle (NVB) in the ear and to provide a substrate for detecting neolymphatic growth,

b) maintaining the mammal under conditions conducive to promoting lymphedema such as allowing the mammal to recover from the surgical manipulation for at least about a few hours, including several hours, up to about one to about five days,

c) administering a candidate compound to the mammal to treat the lymphedema, the administration being prior to, during or after the surgical manipulation, preferably after the surgical manipulation; and

d) monitoring any increase or decrease in ear volume following administration of the candidate compound. Preferred means of measuring ear volume include use of a caliper or conducting a standard water volume assay as provided in the Examples section.

A preferred compound (or mixture of compounds) capable of reducing ear volume in the assay will show at least about a 10% decrease (when compared to a suitable control in which the candidate compound has not been administered), preferably at least about 30% to about 40%, more preferably at least about 50% to about 70% decrease. A preferred example of such a compound is VEGF-2 as well as fragments thereof that give good activity in the rabbit ear assay.

A more specific example of the foregoing rabbit ear assay can be found in the Examples section (sometimes referred to as the rabbit ear model assay or related phrase).

Significantly, the rabbit ear assay can be used to pre-select or screen candidate compounds including allelic variants, fragments, and muteins of VEGF-2 for treating lymphedema in a human patient.

Reference herein to an “effective fragment” or “effective mutein” of a lymphiogenic agent means an amino acid sequence that exhibits at least about 70%, preferably at least about 80% to about 95% of the lymph vessel promoting activity of the corresponding full-length protein as determined by the standard rabbit ear assay. An exemplary effective fragment is a lymph vessel promoting fragment of VEGF-2. Preferred allelic variants and isoforms of VEGF2 will show related activity in the rabbit ear assay.

In some embodiments of the invention, it may be advantageous to combine the lymphangiogenic agents of this invention with at least one angiogenic protein to also promote good blood vessel growth. For example, in embodiments in which the methods of this invention are employed to promote new lymph vessels following trauma, it may also be very useful to promote angiogenesis as well.

Accordingly, the term “angiogenic agent” or “angiogenic protein” refers to any protein, polypeptide, mutein or portion that is capable of, directly or indirectly, inducing the formation of new blood vessels. Folkman, et al., Science, 235:442-447 (1987). Such proteins include, for example, acidic fibroblast growth factors (FGF-1), basic fibroblast growth factors (FGF-2)), FGF-4, FGF-5, vascular endothelial growth factor (VEGF), epidermal growth factor (EGF), transforming growth factor α and β (TGF-α and TFG-β), platelet-derived endothelial growth factor (PD-ECGF), platelet-derived growth factor (PDGF), tumor necrosis factor cc (TNF-α), hepatocyte growth factor (HGF, scatter factor), insulin like growth factor (IGF), IL-8, proliferin, angiogenin, fibrin fragment E, angiotropin, erythropoietin, colony stimulating factor (CSF), macrophage-CSF (M-CSF), granulocyte/macrophage CSF (GM-CSF) and nitric oxidesynthase (NOS). VEGF includes the various forms of VEGF such as VEGF₁₂₁, VEGF₁₄₅, VEGF₁₆₅, and VEGF₁₈₉. See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994).

Preferably, the angiogenic and lymphangiogenic proteins of this invention include a secretory signal sequence that facilitates secretion of the protein. Proteins having native signal sequences, e.g., VEGF, VEGF-2 are preferred. Angiogenic proteins that do not have native signal sequences, e.g., bFGF, can be modified to contain such sequences using routine genetic manipulation techniques. See, Nabel et al., Nature, 362:844 (1993).

The angiogenic action of any given protein, peptide or mutein can be determined using a number of bioassays including, for example, the rabbit cornea pocket assay (Gaudric et al., Ophthalmic. Res. 24:181-8 (1992)) and the chicken chorioallantoic membrane (CAM) assay (Peek et al., Exp. Pathol. 34:35-40 (1988)).

The nucleotide sequence of lymphangiogenic and angiogenic proteins, are readily available through a number of computer data bases, for example, GenBank, EMBL and Swiss-Prot. Using this information, a DNA segment encoding the desired may be chemically synthesized or, alternatively, such a DNA segment may be obtained using routine procedures in the art, e.g, PCR amplification.

In particular, suitable VEGF DNA can be obtained from a variety of sources. For example, one source is the National Center for Biotechnology Information (NCBI)- Genetic Sequence Data Bank (Genbank). A DNA sequence listing can be obtained from Genbank at the National Library of Medicine, 38A, 8N05, Rockville Pike, Bethesda, Md. 20894. Genbank is also available on the internet at http://www.ncbi.nlm.nih.gov. See generally Benson, D. A. et al. (1997) Nucl. Acids. Res. 25: 1 for a description of Genbank.

To simplify the manipulation and handling of the nucleic acid encoding the protein, the nucleic acid is preferably inserted into a cassette where it is operably linked to a promoter. The promoter must be capable of driving expression of the protein in cells of the desired target tissue. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)) and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely effect the metabolism of the organism being treated. The cassette can also be bound to a nucleic acid binding moiety in a synthetic delivery system, such as the system disclosed in WO 95/22618.

In certain situations, it may be desirable to use nucleic acids encoding two or more different proteins in order optimize the therapeutic outcome. For example, DNA encoding two proteins, e.g., two copies of VEGF-2, VEGF-2 and an angiogenic protein such as VEGF or bFGF, can be used, and may in some settings provide benefit over the use of VEGF-2 or bFGF alone. Or a lymphangiogenic protein can be combined with other genes or their encoded gene products to enhance the activity of targeted cells, while simultaneously inducing growth of new lymph vessels, including, for example, nitric oxide synthase, L-arginine, fibronectin, urokinase, plasminogen activator and heparin.

The term “effective amount” means a sufficient amount of nucleic acid delivered to produce an adequate level of the lymphangiogenic protein, i.e., levels capable of inducing the growth of new lymph vessels as determined by the assays described herein and particularly the standard rabbit ear volume assay or a standard lymphoscintigraphy assay. Thus, the important aspect is the level of protein expressed. Accordingly, one can use multiple transcripts or one can have the gene under the control of a promoter that will result in high levels of expression. In an alternative embodiment, the gene would be under the control of a factor that results in extremely high levels of expression, e.g., tat and the corresponding tar element.

For example, an effective amount of VEGF including VEGF-2 as well as muteins and effective fragments thereof can be administered to the mammal at least about 12 hours before exposing the mammal to the conditions conducive to damaging the lymphatic vessels. Such administration can be from about 1 to 10 days before exposing the mammal to the conditions conducive to damaging the vessels if needed.

Alternatively, or in addition, the methods of this invention can include administering the VEGF or VEGF-2 to the mammal following the exposure to the conditions conducive to damaging the vessels.

Reference to a standard lymphoscintigraphy assay means a recognized assay for visualizing lymph vessels using Tc-99 sulfur colloid as a marker. New vessels can be scored by inspection. A preferred lymphangiogenic agent induces the growth of at least about 10% more new vessels, preferably at least about 20% to about 50% more, when compared to a suitable control animal (without administered agent).

Typically, the nucleic acid encoding the angiogenic agent is formulated by mixing it at ambient temperature at the appropriate pH, and at the desired degree of purity, with physiologically acceptable carriers, i.e., carriers that are non-toxic to recipients at the dosages and concentrations employed.

The nucleic acids disclosed herein are preferably introduced into recipient cells of the mammal by any method which will result in the uptake and expression of the nucleic acid by the cells. The introduction can be by standard techniques, e.g. infection, transfection, transduction or transformation. Examples of modes of gene transfer include e.g., naked DNA, Ca₃(PO₄)₂ precipitation, DEAE dextran, electroporation, protoplast fusion, lipofecton, cell microinjection, viral vectors, adjuvant-assisted DNA, catheters, gene guns etc. Vectors include chemical conjugates such as described in WO 93/04701, which has targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses. DNA viral vectors are preferred. These vectors include pox vectors such as orthopox or avipox vectors, herpes virus vectors such as a herpes simplex I virus (HSV) vector [A. I. Geller et al., J. Neurochem, 64:487 (1995); F. Lim et al., in DNA Cloning: Mammalian Systems, D. Glover, Ed. (Oxford Univ. Press, Oxford England) (1995); A. I. Geller et al., Proc Natl. Acad. Sci.: U.S.A.:90 7603 (1993); A. I. Geller et al., Proc Natl. Acad. Sci USA: 87:1149 (1990)], Adenovirus Vectors [LeGal LaSalle et al., Science, 259:988 (1993); Davidson, et al., Nat. Genet., 3:219 (1993); Yang et al., J. Virol., 69: 2004 (1995)] and Adeno-associated Virus Vectors [Kaplitt, M. G., et al., Nat. Genet., 8:148 (1994)].

Pox viral vectors may be preferred in embodiments in which introduction into all cells of the mammal is desired. Avipox virus vectors result in only a short term expression of the nucleic acid. Adenovirus vectors, adeno-associated virus vectors and herpes simplex virus (HSV) vectors are preferred for introducing the nucleic acid into some cells. The adenovirus vector results in a shorter term expression (about 2 months) than adeno-associated virus (about 4 months), which in turn is shorter than HSV vectors. The particular vector chosen will depend upon the target cell and the condition being treated.

Gene guns include those disclosed in U.S. Pat. Nos. 5,100,792 and 5,371,015 and PCT publication WO 91/07487.

If desired, the nucleic acid may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

A particular nucleic acid encoding a lymphangiogenic protein e.g., VEGF-2 is typically introduced by direct injection into the cells (e.g., muscle cells) of the mammal. Such direct injection of the nucleic acid can be prior to, during, or after development of lymphatic condition, particularly lymphedema. A preferred delivery means is a stent, catheter, syringe or related device.

See also U.S. Pat. No. 5,652,225 and Tsurumi, Y et al. (1996) Circulation 94: 3281 for general disclosure relating to administering nucleic acid to a mammal including direct injection methods.

The nucleic acid can be applied topically, for example, painted onto desired tissue surface such as those exposed by surgery. In such a case it is preferable to use a viscous solution such as a gel rather than a non-viscous solution. This may be accomplished, for example, by mixing the solution of the nucleic acid with a gelling agent, such as a polysaccharide, preferably a water-soluble polysaccharide, such as, e.g., hyaluronic acid, starches, and cellulose derivatives, e.g., methylcellulose, hydroxyethyl cellulose, and carboxymethyl cellulose. The most preferred gelling agent is methylcellulose. The polysaccharide is generally present in a gel formulation in the range of 1-90% by weight of the gel, more preferably 1-20%. Examples of other suitable polysaccharides for this purpose, and a determination of the solubility of the polysaccharides, are found in EP 267,015, published May 11, 1988, the disclosure of which is incorporated herein by reference.

By the term “nucleic acid” is meant DNA or RNA including anti-sense DNA or RNA.

Reference herein to a “mammal” is meant to include a rabbit, rodent or a primate. Examples of rodents includes mice and rats. Examples of primates include chimpanzees. A preferred primate is a human.

As discussed, much attention has been focused on understanding the molecular biologic and cellular events surrounding angiogenesis. For example, there is recognition of the angiogenic potential of VEGF-2 in both protein and plasmid forms in the rabbit hind limb ischemic model.

More specifically, VEGF-2 has been reported to bind with high affinity to two endothelial cell receptors: VEGFR-2 (flk-1) and VEGFR-3 (fit 4), the former transducing the biologic angiogenic effect. Fit 4 expression is taught to be limited to lymphatic endothelial cells, and VEGF-2 overexpression in the skin of transgenic mice has been reported to result in hyperplasia of lymphatic vessels and proliferation of lymphatic endothelial cells. The specificity of fit-4 to lymphatic endothelial cells may provide a means for immunohistochemical confirmation of lymphangiogenesis.

The biology of VEGF-2 (sometimes called VEGFC) has been reported. See Olofsson, B. et al. in Current Opinion in Biotech. (1999) 10: 528.

A knock out model has been used to study flt4. For example, one such model has been disclosed as resulting in early embryonic death with numerous blood vascular abnormalities. However, the heterozygote did not display any lymphatic abnormalities. Control of fit 4 expression is believed to be of major importance in embryonic lymphangiogenesis as the receptor, ubiquitous in almost all endothelial cells in early development, later becomes restricted only to lymphatic endothelial cells.

The familial Milroy lymphedema is thought to be related to an fit 4 gene defect, linking this congenital form of lymphedema to a mutation in the filt 4 coding region. The results of this work show that therapeutic VEGF-2 induced lymphangiogenesis. This result may benefit those suffering from lymphedema. In addition, blocking VEGF-2 function either directly or indirectly effect may benefit patients suffering from tumors associated with a proliferation of lymphatic endothelial cells or lymphogenic metastases.

The safety of intramuscular administration of VEGF-2 DNA has been demonstrated in human trials of VEGF-2 gene transfer for therapeutic angiogenesis in critical limb ischemia. No unexpected adverse outcomes were encountered. In fact, it has been found that one patient with critical limb ischemia also had massive lymphedema of his ischemic limb. Post VEGF-2 treatment revealed the exciting finding of an increase in lymphatic drainage vessels on post-treatment lymphoscintigraphy, although extensive vascular disease precluded further clinical follow up.

Animal models of lymphedema have historically been difficult to produce and utilize for in vivo studies, owing to the requisite severe surgical disfigurement of the limb to cause the lymphedema, and to the often rapid regeneration of the divided lymphatics to functional reconstitution. The standard rabbit ear assay (sometimes also referred to herein as the rabbit ear model) according to the present invention, has been shown to reliably produce lymphedema in the operated ear with a more simple and less costly procedure.

All references disclosed herein are incorporated by reference.

The present invention is further illustrated by the following examples. These examples are provided to aid in the understanding of the invention and are not construed as a limitation thereof.

### EXAMPLE 1

### Rabbit Model of Lymphedema

The present study strives to examine the lymphangiogenic potential of VEGF-2 in the rabbit ear lymphedema model. 3 parameters of effect were measured: presence of clinical reduction in ear edema, lymphoscintigraphic evidence of re-establishment of lymphatic outflow in the operated ears, and histologic examination for number and character of lymphatic channels exhibited.

One concern regarding the published rabbit ear lymphedema model is the lack of tissue post-surgery in which to grow new lymphatic vessels. Typically, it is necessary to strip the ear base circumferentially of all soft tissue in order to produce the edema. As such, a modification of the model must first be performed as follows.

Utilizing a bridge of skin overlying the preserved neurovascular bundle to serve as a substrate for neolymphatic growth, an invention objective includes first demonstrating the induction and preservation of clinical lymphedema, and then proceeding with a comparison of VEGF-2 treated operated animals with operated controls regarding edema resolution.

A. Material and Methods.

10 New Zealand White Rabbits, 9-12 months of age, 3-4 kg were used to reproduce the published model and test modification with a skin bridge covering the neurovascular bundle (NVB). Animals were anesthetized, given subcutaneous antibiotics, and both ears shaved. Pre procedural measurements of ear thickness (measured with calipers at a fixed point 7 cm from distal edge of ear) and ear volume (measured by volume of water displacement) were taken.

One ear was operated on, preserving the contralateral ear as a control 1% lidocaine local anesthesia was infiltrated circumferentially around the ear base, and approximately 0.2 cc of Evans blue dye was injected intradermally in the ear tip to visualize the lymphatic channels at their convergence at the NVB. A 2 cm strip of skin, sucutaneous tissue, and perichondrium was surgically removed circumferentially at the base of the ear.

In the region of the NVB (where the lymphatics are known to converge), a dissecting microscope was used to aid in identifying, ligating and dividing, and removing all blue-dyed lymphatic channels for a length of at least 3 cm. Additionally, the artery, vein and nerve were skeletonized of residual soft tissue in case undyed lymphatics were present.

For the first two rabbits, the skin edges were inverted and sutured to the perichondrial edge, leaving at least a 3 cm wide strip of bare cartilage traversed only by the uncovered NVB.

The remaining 8 rabbits in this group were operated on in a similar fashion, however a “skin bridge” was elevated overlying the NVB, which was reapproximated to the divided distal skin edge following excision of all the lymphatics. The skin bridge was intended to provide a substrate for any neolymphatic growth as well as to prevent dessication of the NVB structures.The skin edges were similarly sutured to the perichondrial border, again leaving the 3 cm wide strip of bare cartilage, now traversed by the NVB covered by a skin flap.

All wounds were covered with Xeroform gauze and dry sterile dressing, changed every three days for 1 week, then left uncovered. Rabbits were also maintained on pain medication and antibiotics. Ears were then measured for thickness and volume at days 3, 7, 14, 21, 28 and then monthly.

1. Results of Part A

All 10 rabbits developed significant lymphedema in the operated ear with no change in the contralateral control ear. Followed for at least 90 days, the pattern of edema demonstrated corresponded closely to published model results, with the presence of the skin bridge apparently not effecting restoration of lymphatic drainage. As with the earlier model, the acute phase of edema began immediately, reaching a maximum severity from days 7-14, with slow, gradual resolution (latency period) over the next sixty days.

B. Initial Treatment with VEGF-2

The second part of data gathering focused on gross clinical effect of VEGF-2 administration to the modified model. 8 Rabbits underwent the skin bridge preserving operation and were treated with 500 ug of VEGF-2 DNA injected intradermally in divided doses into the skin bridge itself, as well as into adjacent proximal and distal skin.The injections were repeated every S days for a total of 3 injections. Again ear thickness and volume measurements were made at weekly intervals up to 1 month and then monthly.

1. Results of Part B:

As depicted in the graphs, there was an attenuated initial acute edema phase in the group of rabbits treated with VEGF-2 following lymphatic excision. Graphically there is a trend toward a more rapid return to baseline following the acute edema response than that seen in the control group when both ear volumes and thickness are measured.

Lymphoscintigraphy:

Technecium 99 sulfur colloid was used in 3 normal rabbits to demonstrate a baseline pathway of normal lymphatic egression. 100 microcuries 99Tc was injected intradermally into distal rabbit ear, and scans were performed at 15 minutes and one hour following injection. Additionally, 4 surgical control animals were scanned and demonstrated effective lymphatic outflow blockade out to the one hour time period after radio-labeled administration. These scans have allowed another method of objectively demonstrating successful complete surgical blockade of lymphatic drainage, and lymphoscintigraphy should provide a method of showing time to restoration of lymphatic flow as well as the pathway of the flow. The “dermal backflow”, indicative of lymphatic blockade, was seen, manifested by increased radiocolloid concentration in the distal ear skin.

Histology:

To date, excised lymphatics and three samples of skin bridge tissue specimens have been evaluated using H&E, CD31, and pale staining, and the results have not been definitive. There has been some evidence of numerous lymphatic pathways seen by comparison of pal E and CD31 staining technique, where pal E selectively stains vascular endothelium and CD31 stains all endothelium, including lymphatic endothelial cells.

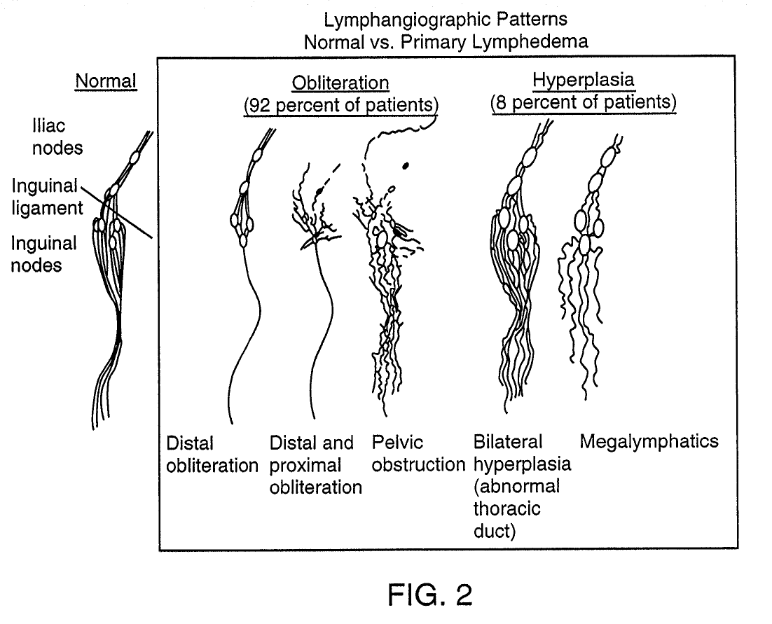

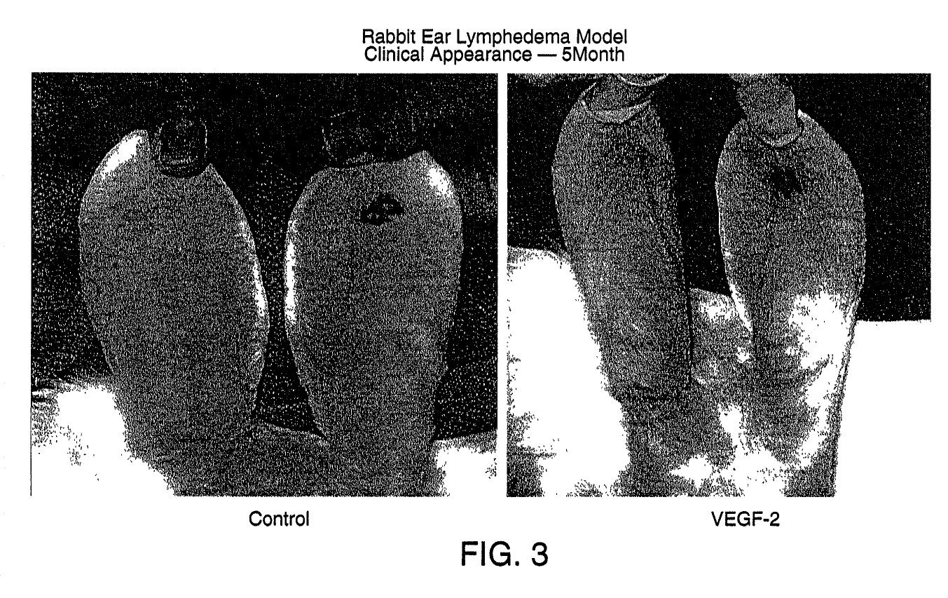

FIG. 3 shows a picture of the rabbit ear lymphedema model. The clinical appearance after five (5) months is demonstrated before and after VEGF-2 DNA treatment. In particular, there is more edema on the left (control) then there is on the right (VEGF-2). The vessels are more easily observed on the right due to the relative lack of lymphedema.

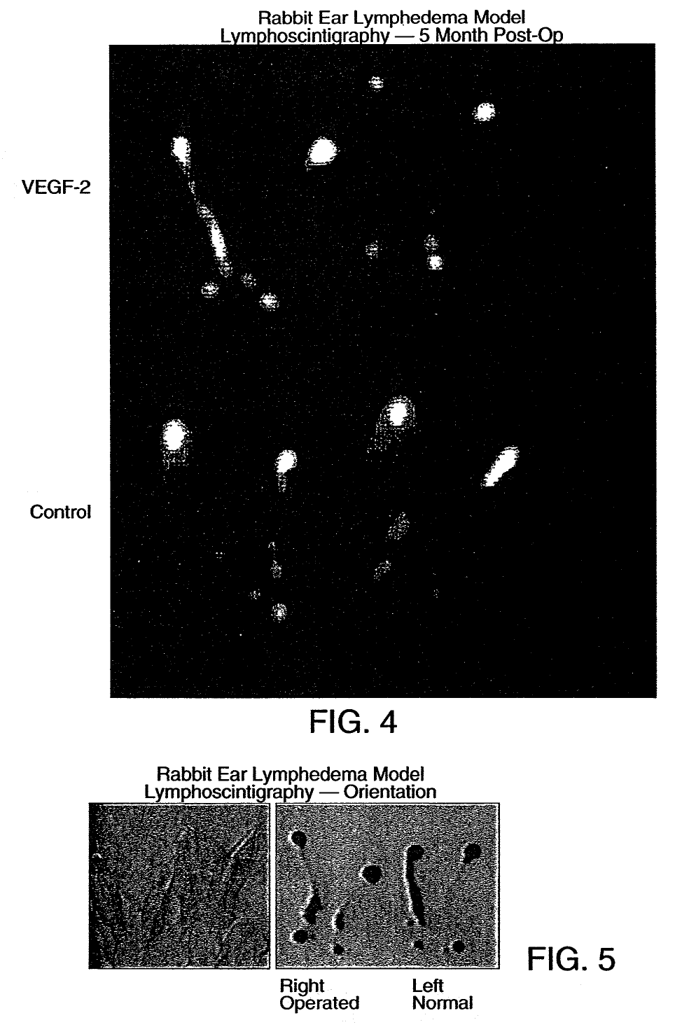

FIG. 4 shows results of lymphoscintigraphy of the rabbit ear model five (5) months post-op. In comparison to VEGF-2 in which there is no so-called dermal back flow, but rather a more linear drainage of the lymphatics with opacification of the nodes (round items at the bottom of the figure). There is in the control much more diffuse opacification of the operated ear so that there is a lot of dermal back flow and no drainage in to the nodes at the bottom of the control figure. In both cases, there are a pair of ears before and a pair of ears afterwards. In the control, the ear to the right in each case was not operated on and is the normal, whereas the one on the left is the one that shows the diffuse nuclear imaging uptake and represents the operated ear with insufficient drainage. For the VEGF-2 images, again there are two pairs with the right ear as you look at the picture in each case serving as the control, whereas the left ear in each case was the operated ear. In the case of VEGF-2, it is difficult to tell the control from the operated and VEGF-2 treated ear.

FIG. 5 is a view of gross photographs of the rabbit ears on the left with the nuclear studies on the right. The description for the nuclear studies is similar to that for FIG. 3, above, except that in this case these both involve VEGF-2 treated ears. Again, there are two ear pairs.

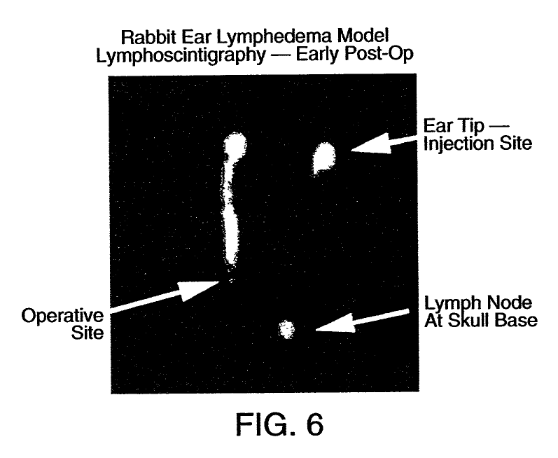

FIG. 6 shows an early post-op image recorded to show the normal ear (the one on the right) and an operated ear (the one on the left); notice again that at this point there is no drainage in to the lymph node at the skull base on the left.

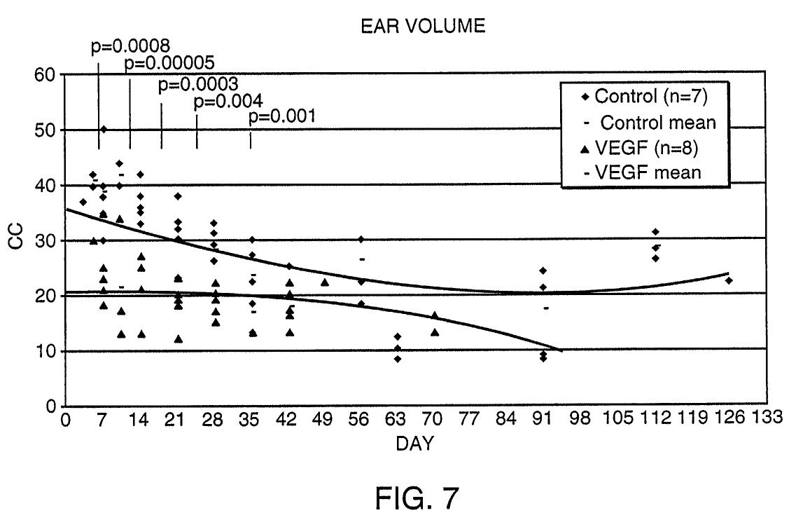

FIG. 7 shows that administered VEGF reduces ear volume in the rabbit model of lymphedema.

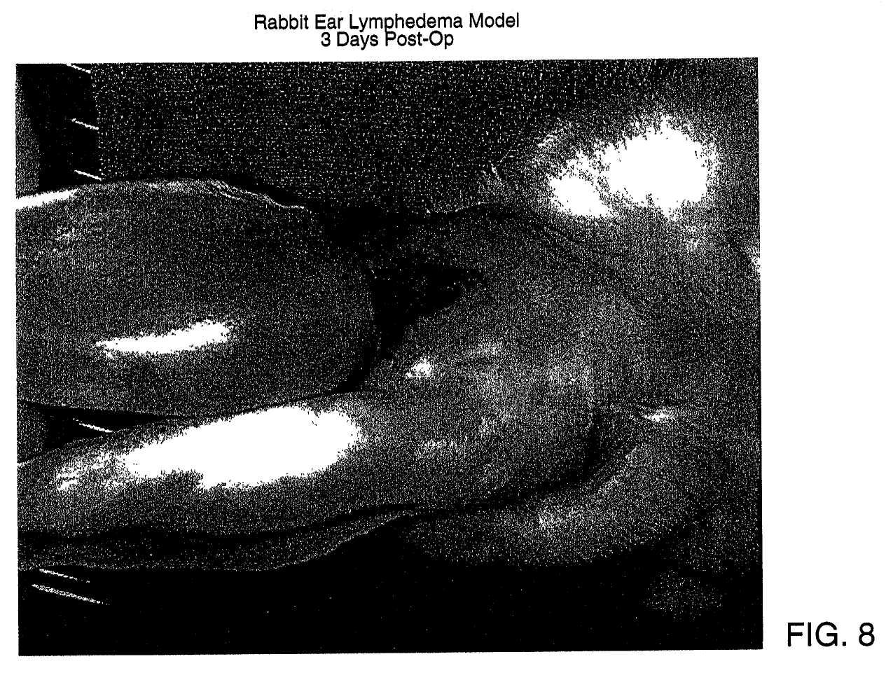

FIG. 8 shows extreme lymphedema in the operated ear in the model. This ear is closest to the top of this photograph. The ear immediately below it has a normal appearance.

### Example 2

### Results of VEGF-2 Gene Transfer in a Novel Animal Model of lymphodema

The results discussed above were repeated and confirmed.

As discussed, VEGF-2 binds with high affinity to endothelial cell (EC) receptors VEGRF-2 (flk-1) and VEGFR-3 (flt-4). Flt-4 expression is primarily limited to lymphatic EC's.

VEFG-2 overexpression in the skin of transgenic mice has been previously shown to result in hyperplasia of lymphatic vessels. As provided above, it was of interest to establish an animal model that could be used to evaluate VEGF-2 gene transfer for lymphangiogenesis in patients with lymphedema whose existing lymphatics are insufficient.

1. Methods

New Zealand White rabbits underwent circumferential excision of skin, soft tissue, and perichondrium of the ear base, preserving a “skin bridge” of tissue to cover the neurovascular bundle (NVB). Under a dissecting microscope, Evans blue-stained lymphatics were ligated and divided, and the artery, vein, and nerve at the neurovascular bundle were skeletonized of surrounding tissue. This created a 2 cm strip of bare cartilage with the skin bridge covering the NVB, preventing dessication and providing a substrate for neolymphatic growth. The unoperated contralateral ear served as control. This surgery was performed in 15 rabbits, 8 of which received 500 ug VEGF-2 naked plasmid DNA injected intradermally in the area of the skin bridge at post-op days 0, 5, and 10. Ear thickness by caliper and ear volume by water displacement measurements were recorded pre-op and weekly thereafter. Lymphoscintigraphy utilizing Tc-99 sulfur colloid was performed post-op to ensure successful surgical blockade of lymphatic egress, and then biweekly.

In this example, all rabbits developed significant post-op lymphedemas; those receiving VEFG-2 gene transfer, however, had statistically significantly reduced ear thickness and volume measurements at each measured weekly time point. Moreover, VEGF-2 promoted a more rapid return to baseline following the acute edema phase. Lymphoscintigraphy subsequently demonstrated classic dermal backflow patterns characteristic of chronic lymphedema; these were obviated by VEGF-2 gene transfer.

These findings characterize a novel animal model of lymphedema, and suggest that VEGF-2 gene transfer may merit clinical investigation for patients with lymphedema.

### Example 3

### Development of Second Generation Rabbit Model of Lymphedema

The results shown in Examples 1 and 2 are most encouraging. It is possible to extend the results by developing second generation rabbit models to confirm analysis of the lymphoscintigraphical information.

In one approach, it is possible to perform a complete surgical block of lymphatic flow from the ear, followed by assignment to control or VEGF-2 treated groups. These two groups can be followed longitudinally, undergoing measurement of ear thickness and volume, repeat lymphoscintigraphy at 1 week intervals to demonstrate any new lymphatic growth across the tissue bridge region and subsequent sacrificing of 2 rabbits from control and treatment groups at 2 week intervals for histologic examination.

An especially useful second generation rabbit model is one in which flt4 antibody staining is employed to provide a more definitive marker specific for lymphatic endothelium. Although an assay for measurement of blood VEGF-2 levels is currently not available, it is possible to collect the rabbit blood samples at weekly intervals for storage until such an assay is available.

### Example 4

### VEGF-2 Gene Transfer Promotes Lymphangiogenesis in Patients

Intramuscular gene transfer of naked VEGF-2 DNA was performed on a patient suffering from lymphedema. The gene transfer was performed on skeletal muscle in the patient to promote lymphatic development and treatment of lymphedema. Radioisotope studies documented improved lymphatic drainage in the patient.

Briefly, the VEGF-2 naked DNA was directly injected into the skeletal muscle. For applications involving a human limb exhibiting lower extremity edema, eight (8) injections are required of 8 mg of the DNA ever two (2) weeks. The injection protocol can be repeated as needed including three times.

It will be appreciated that different dose strategies may be required depending on recognized parameters such as the overall health of the patient, sex, type and severity of the lymphedema and the like.

In addition, treatment of some patients may require use of one or more viral vectors that encode the VEGF-2 DNA as described above.

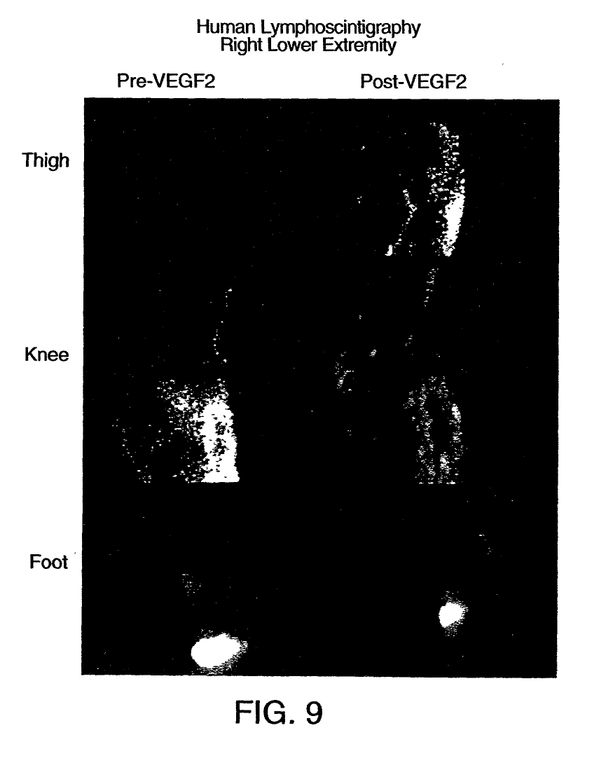

FIG. 9 shows results of treating a human patient along lines discussed above. In the post-VEGF-2 picture, linear streaks in the middle image on the right represent new lymphatic channels that have formed. None of these can be seen on the nuclear image on the left (pre-VEGF2).

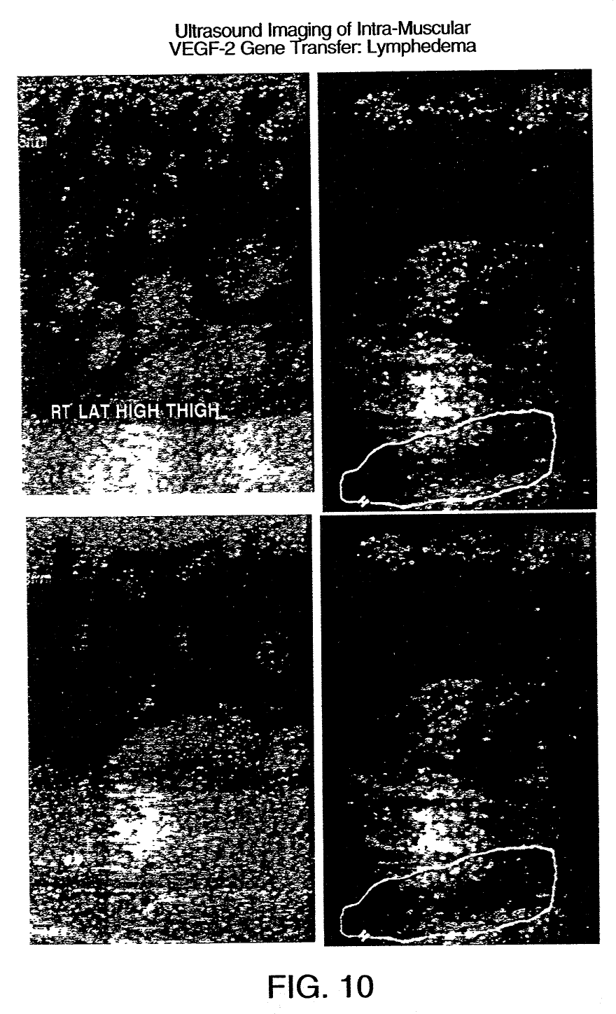

FIG. 10 are ultrasound images demonstrating the extent of edema in the patient whose scintigraphy was shown in FIG. 9.

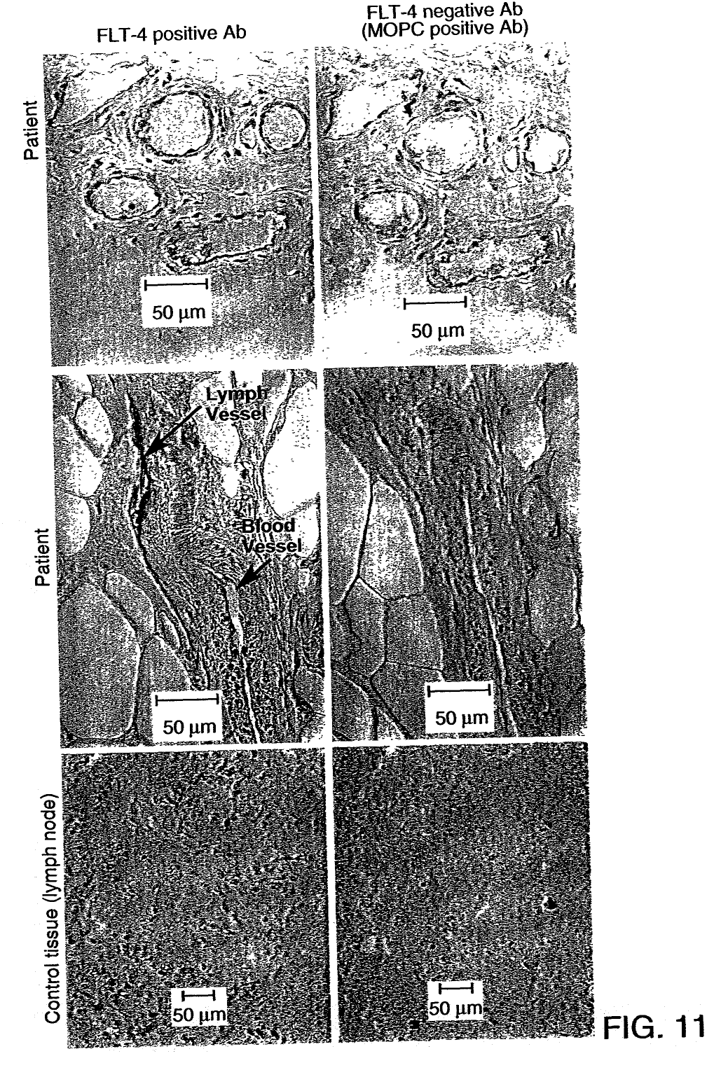

FIG. 11 shows specific antibody staining for lymphatic vessels in the patient shown in FIG. 1 following VEGF-2 gene transfer. Although it is interesting that we see the lymphatics, it is acknowledged that the data in this figure cannot distinguish between lympatics formed pre-and post VEGF 2 gene therapy.

### Example 5

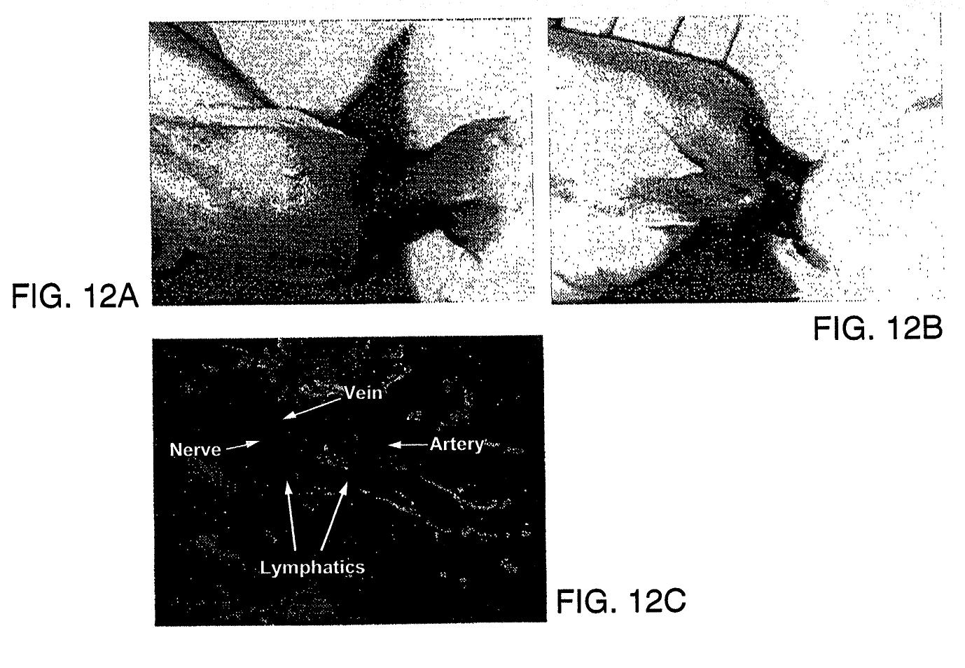

### Rabbit Ear Model (See FIGS. 12A-C)

Results of the prior examples were repeated and extended.

New Zealand White rabbits with the age of 3.5 to 4.5 yrs were used. Anesthesia was obtained by intramuscular injection of ketamine(80 mg/kg) and xylazine(20 mg/kg) and supplemented as required. Additionally, 0.15 mg of buprenorphine was administered intramuscularly and 3 ml of 1% lidocaine were injected around the base of the ears. Antibiotics Enrofloxacin(7 mg/kg) was administered subcutaneously 30 minutes before operation and daily for 14 days. The right ear was operated in all animals. Before the operation, the lymphatic vessels were identified by injection of 0.2 ml of 1% Evans blue intradermally at the dorsal tip of the right ear. The left ear was preserved to be used as a negative control.

About 3-cm wide strip of skin, subcutaneous tissues and perichondrium were circumferentially excised from the base of the ear, except for the central portion(1 cm width) of the dorsal skin named skin bridge underneath which runs the neurovascular bundle. After distal edge of the skin bridge was incised and subcutaneous tissues were dissected to the proximal edge of the skin bridge, the skin bridge was flipped over. Under a dissecting microscope, Evans blue-stained lymphatic channels and plexuses were carefully dissected from surrounding tissues and the lymphatic stumps were resected after ligation. The central artery, vein, and nerve at the neurovascular bundle were isolated from surrounding tissues, a process described as skeletonization. After removing all other tissues beneath the skin bridge, the skin bridge was reapplied to the distal skin. Other edges of skin were inversely sutured to the border of perichondrium with 6.0 prolene to prevent reapproximation of skin edges and recanalization of the lymphatics. This created at least 3 cm-strip of bare cartilage providing a substrate for neolymphatic growth. See FIGS. 12A-C.

Establishment of a Rabbit Ear Model

To determine the effect of administered lymphogenic growth factor on lymphedema, we sought to establish an appropriate animal model. Various rabbit ear models were modified for our purpose. Ear thickness and volume was used to physically assess the degree of lymphedema, and lymphoscintigraphy was used for functional evaluation. Initial experiments showed that in case of young(6-8 month old) New Zealand white rabbits, though they developed significant lymphedema after the surgery, the course of lymphedema regression was so fast not to properly assess the effect of gene transfer. In case of old rabbits(3.5 to 4.5 years old) used in our experiments, significant lymphedema was developed immediately after the surgery, and sustained for more than 12 weeks. Additionally, lymphoscintigraphy at 12 weeks showed dermal backflow pattern and faint visualization of skull base lymph nodes in most cases, confirming that lymphatic dysfunction existed until that time point.

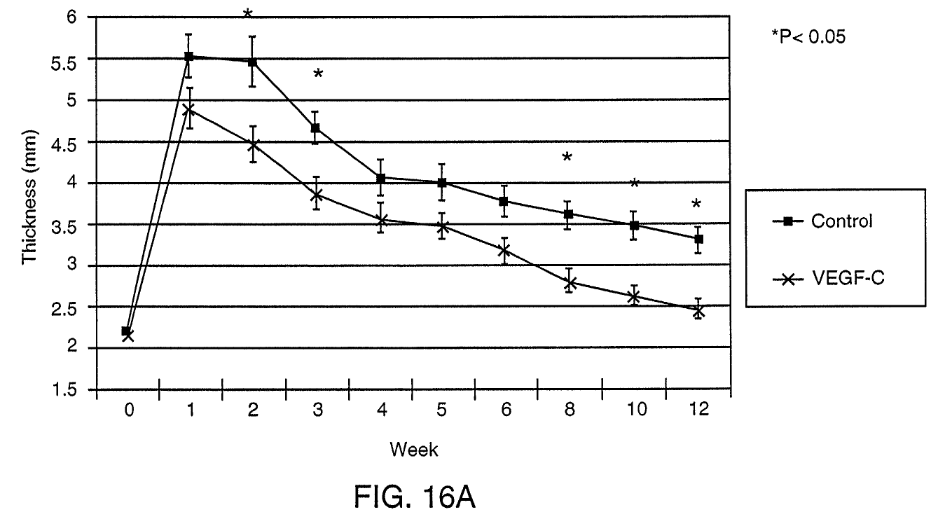

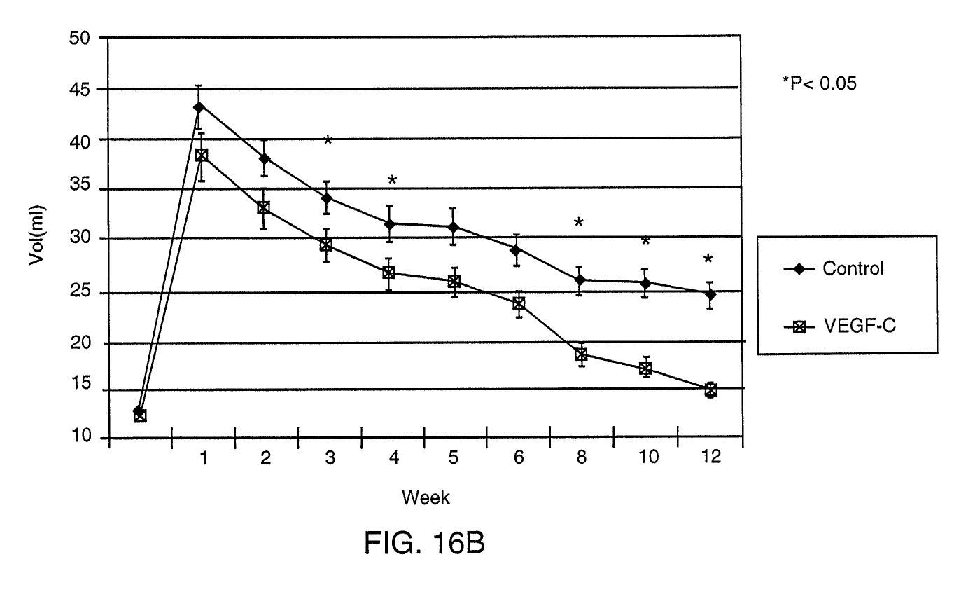

Thickness and Volume Measurements. See FIGS. 16A-B.

To investigate the effect of VEGF-C gene transfer on lymphedema, we measured ear thickness and volume over 12 week period. The time course of ear thickness of the operated ears showed consistent differences between the control and VEGF-C treated groups at every time points until 12 weeks. Statistical analysis disclosed significant differences at weeks 2 and 3, that persisted at 8, 10, 12 weeks. Again, the time course of ear volume measured by the water displacement method showed consistent differences between the control and VEGF-C groups at every time points. The detumescence was readily evident in the VEGF-C treated ear over the time course and the volume measurements disclosed significant differences weeks 2 to 4 and 8 weeks thereafter between the groups.

### Example 6

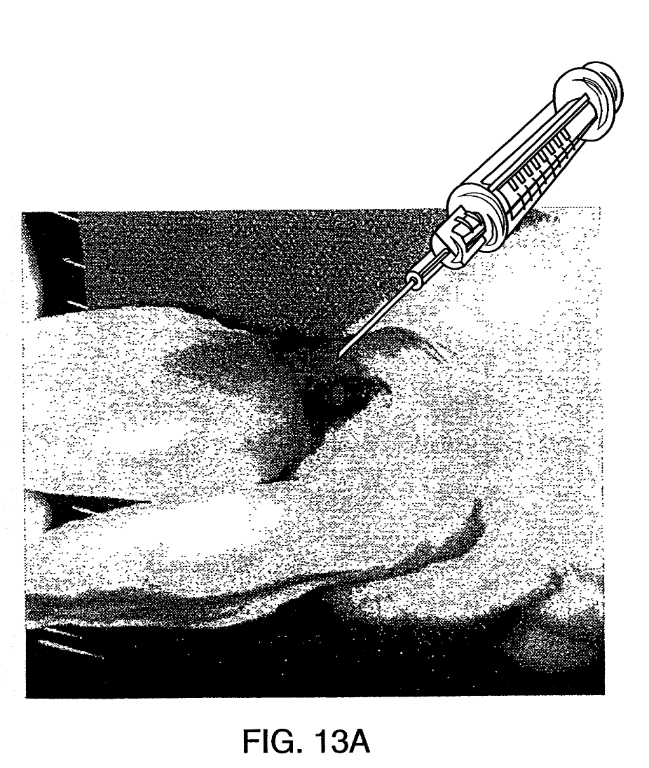

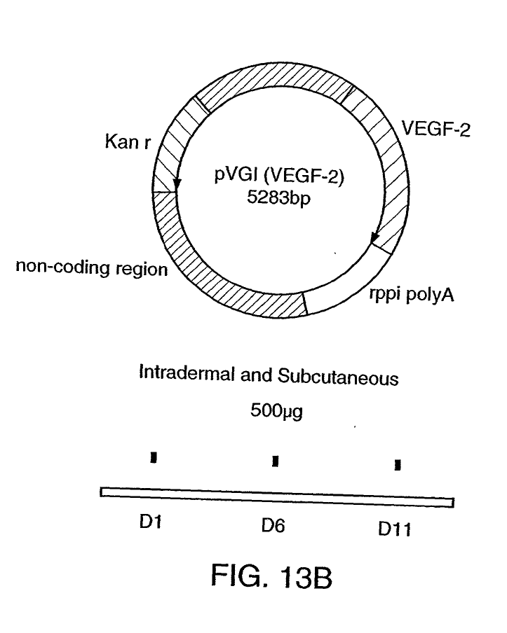

### Gene Transfer Protocol in Rabbit Model. See FIGS. 13A-C.

Total 24 rabbits were randomized into two groups in a blinded fashion before operation. One group served as control and the other group as the VEGF-C gene transfer group. In the VEGF-C treated group, 500 μg of phVEGF-C in 0.5 ml volume was injected intradermally and subcutaneously, at and around the skin bridge using a 27-gauge needle post-operative days 1, 6 and 11, respectively. In the control group, the same volume of saline was injected in an identical fashion.

Thickness and Volume Measurement

Both ears were shaved to facilitate measurements of thickness and volume. In both experimental groups, the ear started to swell only a few hours after surgery. The increasing thickness of the rabbit ears, was measured at the point 1 cm medial and distal from the medial border of the skin bridge with a vernier caliper. Water displacement measurements were carried out for evaluating the volume of both the operated and unoperated ears. The ear was put in a 50 ml cylinder filled with water. After removing the ear, the overflown water in the saucer was measured and used as the volume of the ear. The extent of the measurement from the tip of the ear was made equal for both ears and every time points. The thicknes and volume was measured before surgery and every week until 6 weeks and thereafter every two weeks until 12 weeks.

Microscopic Measurement of Ear Skin Thickness

Thickness of the ear skin was measured under a microscope in cross section of the skin bridge after trichrome staining 6 weeks after the surgery. The net skin thickness was defined as the distance from the surface of the skin to the upper margin of the ear cartilage.

Lymphoscintigraphy and Quantitative Analysis

Preparation of Filtered Technetium-99m-Sulfur Colloid

Technetium 99m-sulfur colloid(Tc-99m-SC) was prepared using Cis-Sulfur Colloid kit(CIS-US, Inc., Bedford, Mass., USA) and Tc-99m generator, Ultra-TechneKow DTE(Mallinckrodt Medical, Inc., St. Louis, Mo., USA) according to manufacturer's instructions. The final preparation was filtered through a sterile 100 nm filter(Millex-VV, Millipore Corp., Bedford, Mass., USA)(26). This filtered sulfur colloid preparation was used for lymphoscintigraphic studies. Tc-99m-filtered SC was injected intradermally to the dorsal tip of both ears of anesthetized rabbits at a dose of 50 μCi in a volume of 0.1-0.2 ml using insulin syringe with 27-gauge needle.

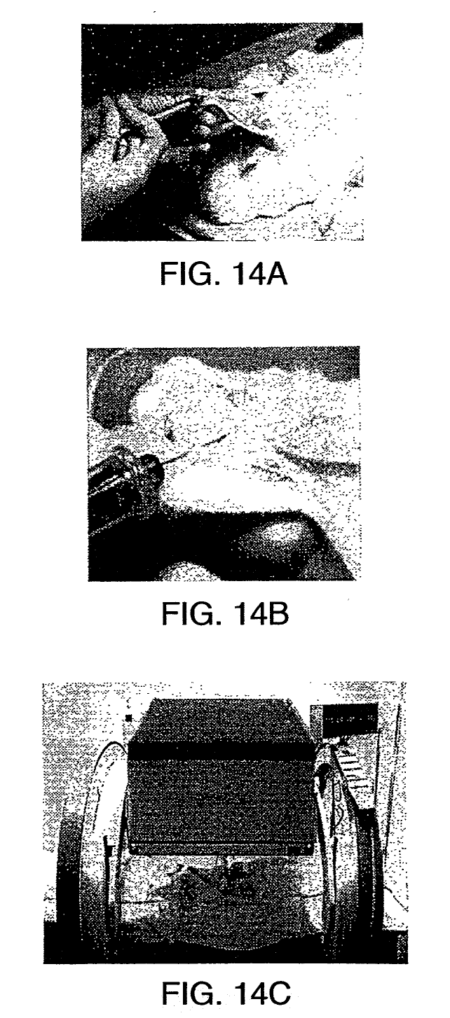

Lymphoscintigraphy See FIGS. 14A-C.

Imaging was performed using a large-field-of-view gamma camera(Genesys, ADAC, Milpitas, Calif., USA) interfaced with a dedicated workstation system and low energy, multipurpose parallel-hole collimator with a 20% window centered over the 140 keV photopeak. Images were obtained 15 minutes and 1 hour after injection with a 5-minute scanning time and onto a matrix size of 128×128×16. The images included the whole ear and base of the skull. Images were digitally stored in order to quantify the level of radioactive material within the ear. Data acquisition process was identical in all rabbits. Imaging of ears was performed at postoperative day 1 to ensure successful surgical blockade of lymphatic egress, and then 4, 8 and 12 weeks. Animals were kept anesthetized for the duration of the imaging sessions.

FIGS. 14A-C are described in more detail as follows. The figures show reliable and reproducible methods for confirming lymphedema and for assessing functional status of lymphatic systems. Particularly, the figures exemplify intadermal injection of Tc^{99 m}-sulfur colloid with 27 guage needle. Early (15 min) and delayed (60 min) images were taken with a gamma camera. Radioactivity was measured in both operated and normal ears excluding the injection site. The ratio of operated vs. normal ear was compared between VEGF-C and control groups.

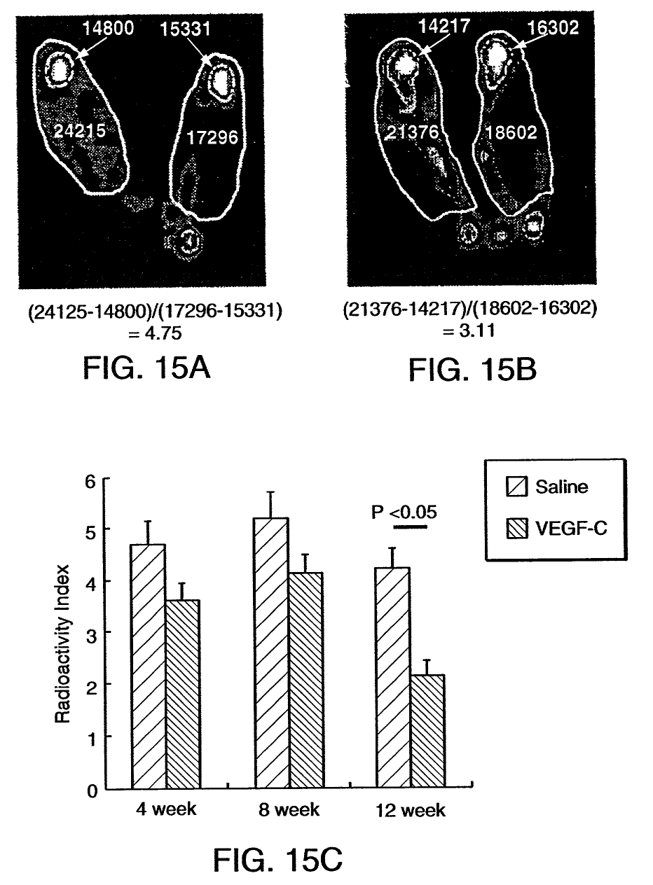

Quantification of Imaging See FIGS. 15A-C.

To quantitatively compare lymphatic drainage of the injected radiotracers, radioactivity within the rabbit ears were counted by an observer blinded to the treatment group. For this quantification, it is assumed that for a given rabbit, lymphatic draining capabilities are the same for both ears. Same doses of radioisotopes were injected at the tip of both operated and intact ears. With use of workstation system(Pegasys ver 3.4, ADAC lab., Milpitas, Calif., USA), radioactivity was measured in 1-hour delayed images. In order to avoid the high concentration of radioactivity at injection sites, we subtracted gamma counts at injection sites from the remainder of the ear, which was used as the remaining radioactivity of the ear. For standardization, the radioactivity ratio of operated vs normal(contralateral) ear, named radioactivity index(RAI), was used to compare radioactivity between VEGF-C and control groups at weeks 4, 8 and 12, respectively.

In summary, it was found that gene transfer of VEGF-C reduces lymphedema in a rabbit ear model.

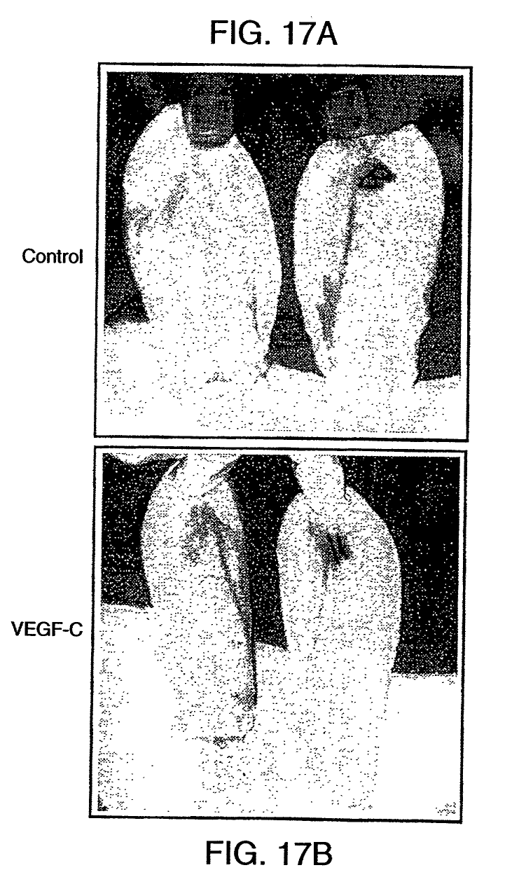

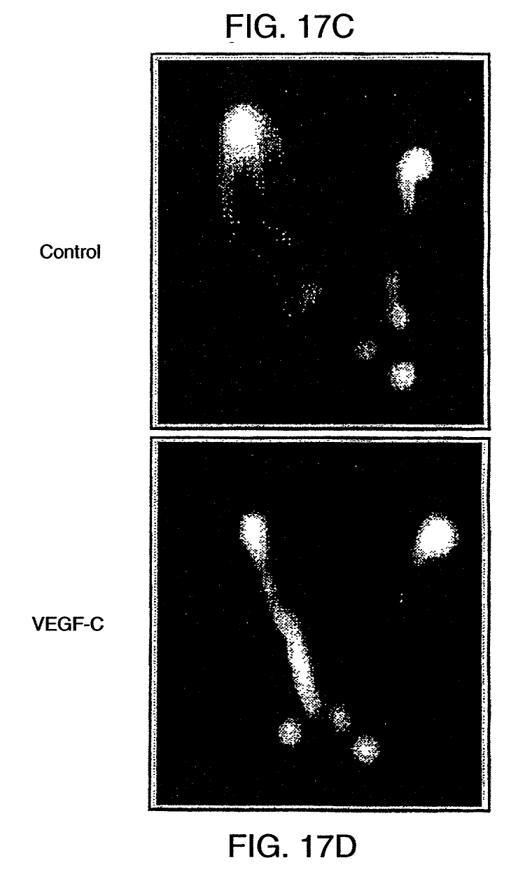

Gross examination See FIGS. 17A-D

Even on long-term follow-up gross examination at 5 month, compared with the normal ear, the operated ear from control group, appeared more voluminous and the underlying vessels were less conspicuous due to fibrotic changes in subcutaneous tissues. However the operated ear from VEGF-C treated group appeared similar to its normal counterpart.

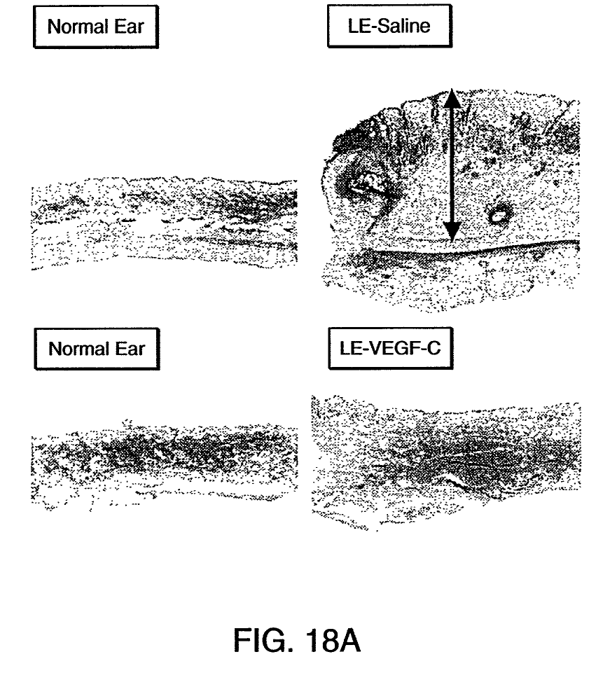

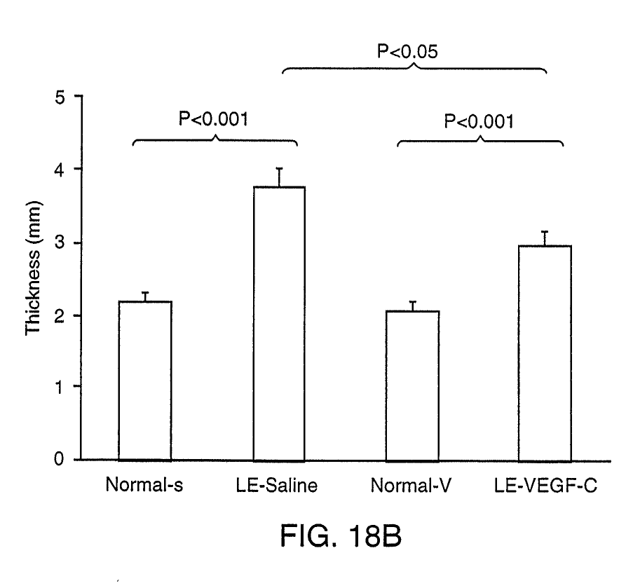

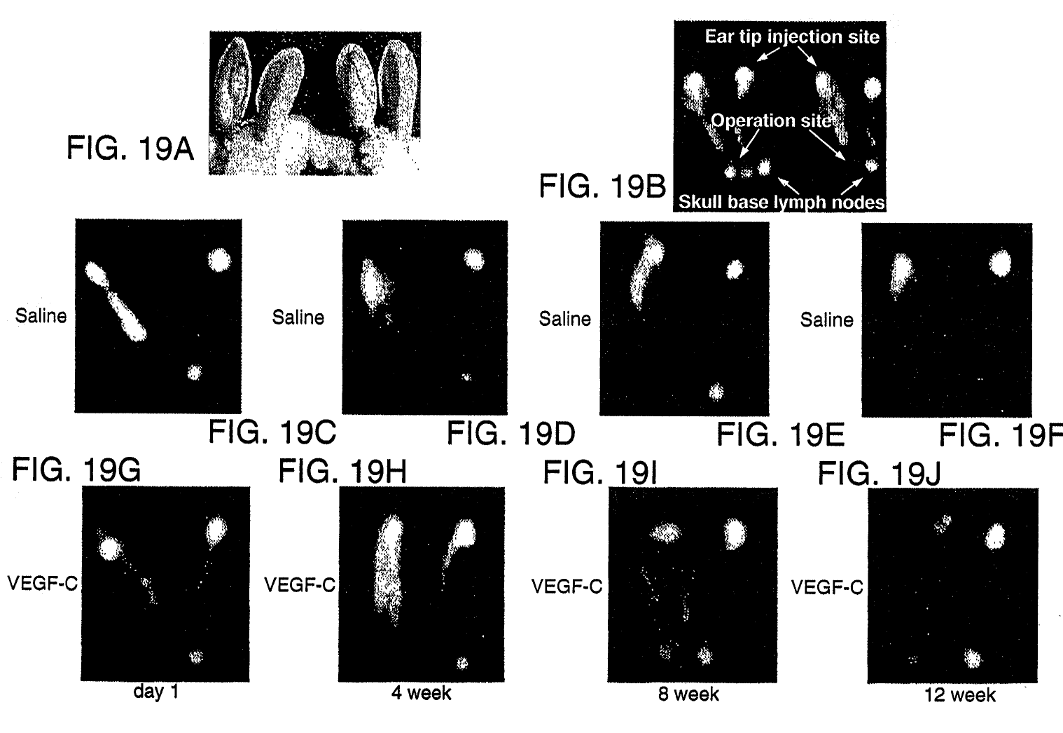

Microscopic Assessment of Skin Thickness See FIGS. 18A-B

To better delineate the effect of VEGF-C on the ear skin thickness, ear skin thickness was compared at 6 week histologic section under a microscope, which is more accurately reflecting the fibrotic changes in the later stage of lymphdema. Compared with normal counterpart, the operated ears from both groups showed significantly greater skin thickness. However the VEGF-C treated group showed significantly smaller skin thickness compared with saline-injected group.

It was found that gene transfer of VEGF-C improved lymphatic dysfunction in a rabbit ear model of lymphedema.

Lymphoscintigraphic Findings See FIGS. 19A-J.

In normal ears, lymphatic flow assumes a linear pattern and the draining lymph nodes are clearly visible at the base of skull. With use of this standardized protocol, normal lymphatic flow was recognized by detection of symmetric radiotracer uptake in the skull base lymph nodes within 15 minutes after injection. A transit time of more than 15 minutes indicated delayed lymphatic transport. After lymphedema operation, the lymphatic passages were blocked, trapping the outflow of radiotracers with prevention of the tracers from reaching the lymph nodes and pressure overloaded lymphatic flow go backward along the normally unvisible dermal lymphatic networks. Imaging performed at postoperative day 1 showed successful surgical blockade of lymphatic egress in all animals. Follow-up lymphoscintigraphy at 4, 8 and 12 weeks showed increased radiotracer clearance from the operated ears over the time course, which was more efficient in VEGF-C treated ear compared with saline-injected ear. Long-term follow-up images revealed the lymphedematous ear from the control group still shows typical dermal backflow pattern without visible lymph node uptake while the ear treated with VEGF-C shows a linear pattern of lymphatic drainage similar to its normal counterpart, including flow into draining lymph nodes at skull base.

Quantitative Analysis of Lymphoscintigraphy See FIGS. 15A-C.

To quantitatively determine the efficiency of lymphatic drainage, we compared the remained radioactivity within the treated ear with use of radioactivity index. Higher values of this index indicate more persistent radioactivity, and consequently less lymphatic drainage from the rabbit ears. At each time points of 4, 8, 12 weeks, radioactivity indices were lower in the VEGF-C treated group than in the saline-injected group, achieving a statistically significant difference at 12 weeks follow-up(4.2±0.5 vs 2.15±0.4, p <0.05). These findings imply a greater lymphatic drainage for the VEGF-C treated group.

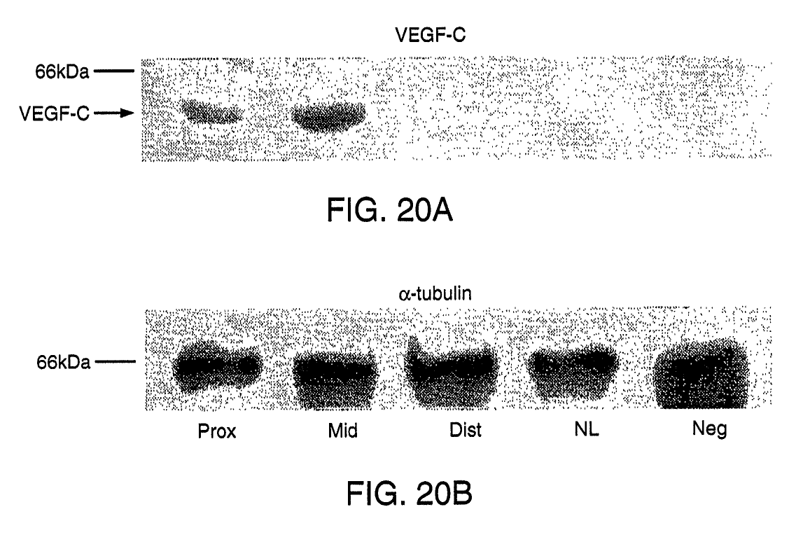

Transgene Expression of phVEGF-C in a Rabbit Ear Model See FIGS. 20A-B.

To assess the levels of VEGF-C protein expression and the results of transgene expression in this study, we performed Western blotting for VEGF-C expression in ear skin. The molecular mass of VEGF-C polypeptide is reported to range from 15 kDa to 58 kDa according to the processed state (27). In our experiments, 58 kDa band were detected with use anti-VEGF-C antibodies, which corresponds to the most unprocessed form. Specificity controls were made with samples from the bridge of VEGF-C treated ear in which the primary antibody reaction was skipped and the Western blot was performed. Densitometric analysis of multiple experiments performed on samples from 4 different animals per group revealed that VEGF-C protein expression at and around the the skin bridge from VEGF-C treated rabbit was significantly higher than the normal contralateral ear or saline injected skin bridge from control group p<0.05)

It was found that VEGFR-3 expression is increased after gene transfer of VEGF-C.

Preparation of phVEGF-C

The VEGF-C plasmid used for this study, named phVEGF-C, is a 5283 base pair plasmid that contains the human VEGF-C coding sequence. Expression from the VEGF-C gene is modulated by the presence of enhancer sequences from cytomegalovirus and promoter sequences of the Rous sarcoma virus. Ribonucleic acid (RNA) processing signals (rat pre-proinsulin polyadenylation and 3′ splice sequences) are present to enhance VEGF-C messenger RNA stability. The plasmid also contains a gene that confers kanamycin resistance to the host cells.(Schratzberger et al, 2000)

### Example 7

### Western Analysis of VEGF-C Transgene Expression in Tissue

Samples harvested from the skin bridge and proximal and distal to the skin bridge of the operated ears and from the bridge site of the contralateral ears, were snap frozen in liquid nitrogen 7 days after the second injection of phVEGF-C (post-operative day 13), respectively. Samples were homogenized in lysis buffer (100 mM potassium phosphate, 0.2% Triton X-100) supplemented with a protease inhibitor cocktail (Roche, Mannheim, Germany). Total protein extracts were quantified by the BCA protein assay kit (Pierce, Rockford, Ill.). Protein extracts (100 μg per sample) were separated on a 12% SDS-PAGE(Ready Gels, Bio-Rad, Hercules, Calif.) and electrotransfered onto PVDF membranes(Hybond-P, Amersham Pharmacia Biotech, Piscataway, N.J.), which were blocked overnight with 5% nonfat dry milk in 0.2% Tween PBS (T-PBS). Samples were probed with a VEGF-C goat polyclonal antibody (Santa Cruz Biotechnology, Santa Cruz, Calif.; 1:500). The membrane was washed 3 times in T-PBS and then incubated with horseradish peroxidase-conjugated anti-goat IgG(1:5000) for 1 h. Antigenantibody complexes were visualized after incubation for 1 min with ECL+chemiluminescence reagent (Amersham Pharmacia Biotech) at room temperature, followed by exposure to Hyperfilm ECL (Amersham Pharmacia Biotech). Equal protein loading among individual lanes was confirmed after stripping the membranes with ImmunoPure elution buffer (Pierce) by reprobing the membranes with an α-tubulin mouse monoclonal antibody(Calbiochem, San Diego, Calif.; 1:1000 dilution). We performed the same procedure using VEGF-C mouse monoclonal antibody(Human Genome Science, Rockville, Md.; 1:500)and horseradish peroxidase-conjugated anti-mouse IgG. Each experiment was repeated at least three times with different cellular extracts. Densitometric analysis was performed (NIH imaging program) to allow for quantitative comparison of protein expression. Results shown are representative of 3 to 5 experiments.

### Example 8

### Molecular Cloning of a Partial Rabbit VEGFR-3 cDNA

Because the rabbit VEGFR-3 DNA sequence has not been disclosed, we sequenced part of the VEGFR-3 CDNA using degenerate oligonucleotides. Degenerate oligonucleotides were designed from conserved aa sequences NVSDSLEM and WEFPRER, located 90 aa residues upstream or 40 aa residues downstream, respectively, of the trans-membrane domain of human and mouse VEGFR-3/Flt-4(Finnerty et al 1993, Galland 1993). The deduced oligonucleotide sequence were 5′-AACGTGAG(CT)GACTC(GC)(CT)T(AGCT)GA(AG)ATG-3′ and 5′CC(GT)YTC (CT)C(GT) GGG(AG)AA(CT)TCCCA-3′, respectively. Total RNA was extracted from kidney, ear, paraaortic lymph nodes, mesentery, and lung using TRIzol(Life Technologies, Inc., Grand Island, N.Y., USA) according to the standard acid-guanidium-phenol-choloroform method. Two microgram of total RNA were reverse transcribed using random hexamer and Moloney murine leukemia virus reverse transcriptase(MMLV-RT) (SuperscriptII™, GibcoBRL, Life Technologies, Inc., Grand Island, N.Y., USA) according to the manufacturer's instructions. Briefly, the RNA was reverse transcribed in 201 of reaction mixture containing of 10 mM of each DATP, dCTP, dGTP, and dTTP; 0.1M DTT; 200U MMLV-RT, 40U Ribonuclease inhibitor and buffer. One tenth volume of the reverse transcriptase(RT) product was subjected to polymerase chain reaction(PCR) in the presence of the above-mentioned pair of oligonucleotides and Taq DNA polymerase(GibcoBRL). PCR cycles were as follows: 94° C., 2 min(once); 94° C., 15 sec; 50° C., 30sec; 72° C., 1 min(30 times); 72° C., 10 min(once). A single PCR product of approximately 470 base pairs was obtained from all the tissues The PCR product from the kidney sample was subcloned into the pBluescript vector(PCR-Script Amp Cloning Kit, Stratagene, La Jolla, Calif., USA) for sequencing and probe preparation. Sequencing was performed utilizing simultaneous bidirectional-sequencing technique using Sequwncher(GeneCodes, Ann Arbor, Mich.)(MWG Biotech Inc., High Point, N.C., USA)

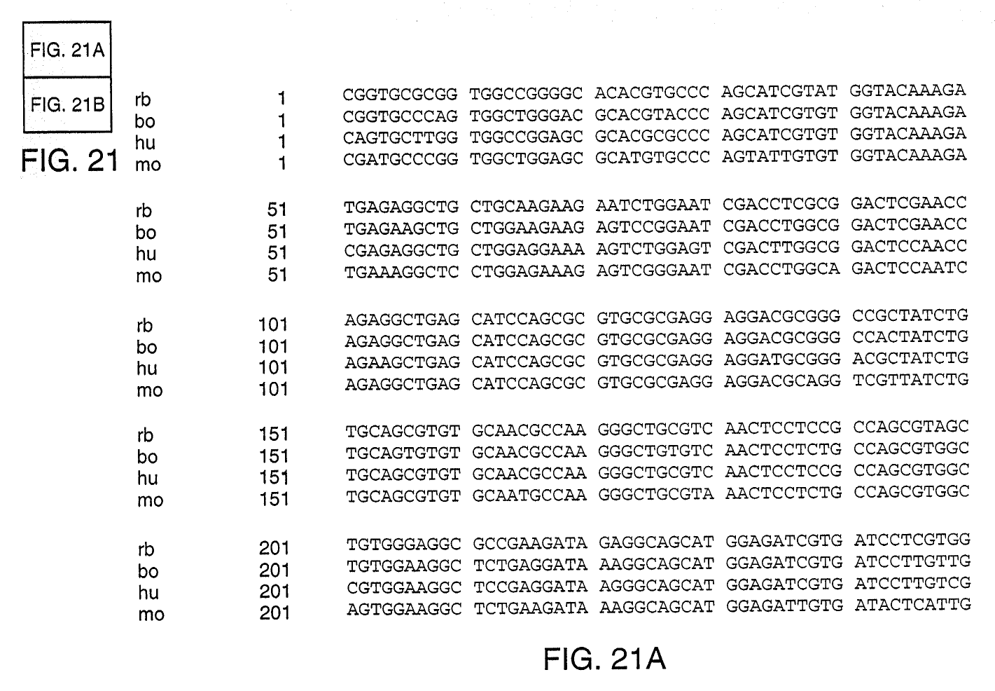

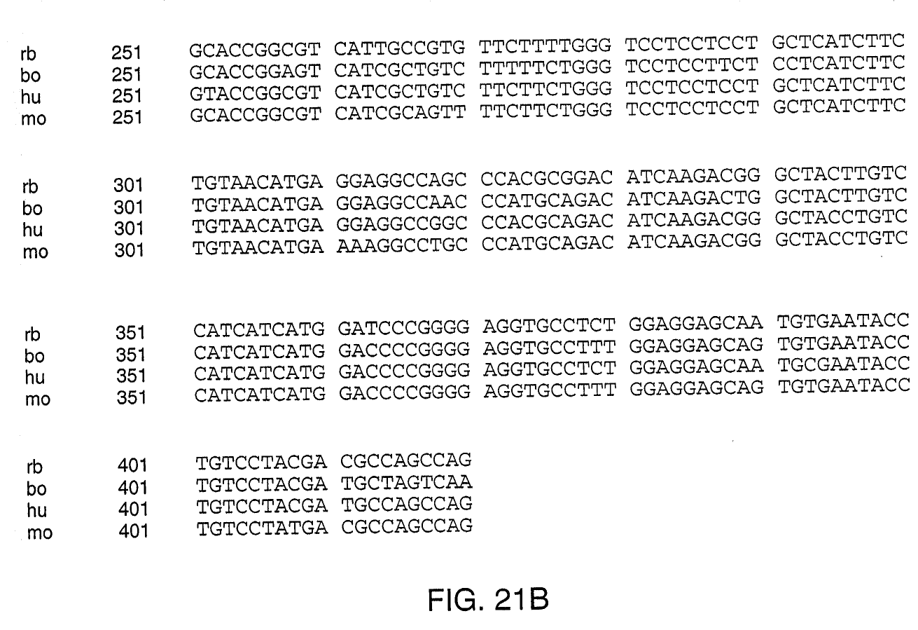

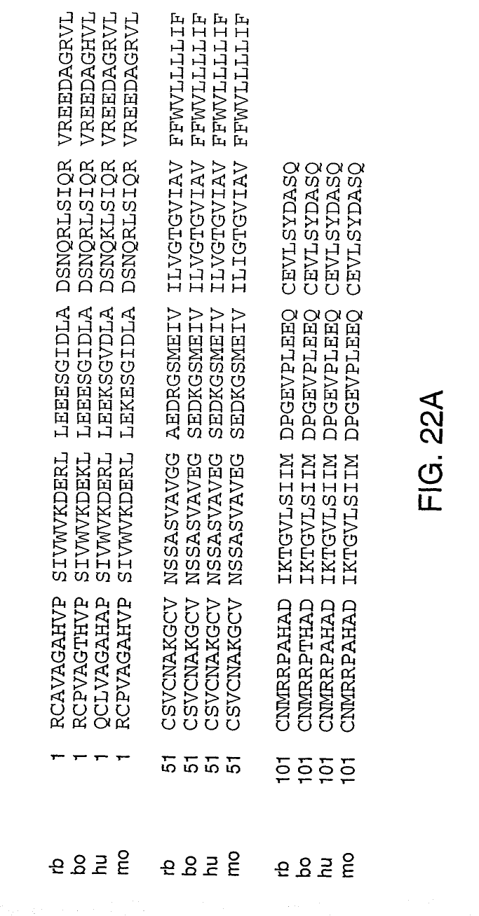

Cloning of a Partial Rabbit VEGFR-3 cDNA See FIG. 21.

A partial 420-base pair rabbit VEGFR-3 cDNA was cloned by RT-PCR from adult rabbit kidney using degenerative oligonucleotide primers. The cDNA is derived from the VEGFR-3 coding sequence and spans the transmembrane domain. At the nucleotide level, the cDNA displayed 90.5% and 87.9% identity with the same region of human and mouse VEGFR-3, respectively. At the protein level, the rabbit VEGFR-3 clone displayed 92.9% and 94.3% identity with human and mouse VEGFR-3, respectively.

FIG. 21 is explained in more detail as follows. Degenerate oligonucleotides designed from conserved amino acid sequences NVSDSLEM and WEFPRER, located 90 amino acid residues upstream or 40 amino acids downstream of the transmembrane domain of human and mouse VEGFR-3 were obtained. Reverse transciption and PCR were conducted. The resulting RT-PCR product was subcloned into pBluescript vector for sequencing and prope preparation. The product had a molecular weight of about 470 bp as estimated by polyacrylamide gel electrophoresis.

### Example 9

### Quantitative RT-PCR Analysis of VEGFR-3

At postoperative day 14, samples were harvested from the bridge site of both ears. Total RNA was isolated using Totally RNA(Ambion, Austin, Tx., USA) according to the manufacturer's instructions. The RT was followed by a PCR reaction conducted in a total volume of 50 μl that contained 1.5 mM MgC12, 10 mM of each DATP, dCTP, dGTP and dTTP; 0.4 Units of Taq DNA polymerase(GibcoBRL). The primer pair used, designed on the basis of the coding cDNAs for rabbit VEGFR-3(this article) was: for sense 5′-TATGGTACAAAGATGAGAGGC-3′, and for antisense 5′-ACAGGTATTCACATTGCTCCT3′. The PCR with this pair of primer yielded 362 bp reaction product, and was tested with cDNAs of various rabbit tissues(lung, liver, mesentery, lymph nodes) to test the specificity before proceeding to the quantitative RT-PCR. In order to quantify the VEGFR-3 mRNA product in both VEGF-C treated and control ears, we used the “competimer” quantitative PCR technique: VEGFR-3 cDNA and 18S cDNA were co-amplified at the same time for each sample. In the same mix with VEGFR-3 PCR we added a mix of 18S primer pair/18S 3′-end modified primers(competimers) at a ratio of 1/9(Ambion, Austin, Tx.), yielding a 488-bp product. After forty cycles of PCR with the above condition, PCR products were separated on agarose gel containing ethidium bromide and quantified by using integrated density analysis software(EagleSight Software 3.2, Staratagene, La Jolla, Calif., USA). RT-PCR and relative quantification of PCR products were performed at least three times on samples from both treated and contralateral ears(n=5 in each group).

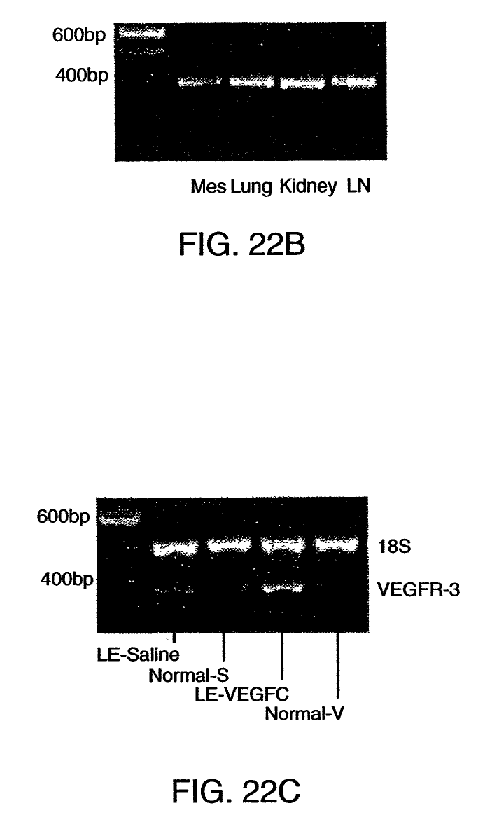

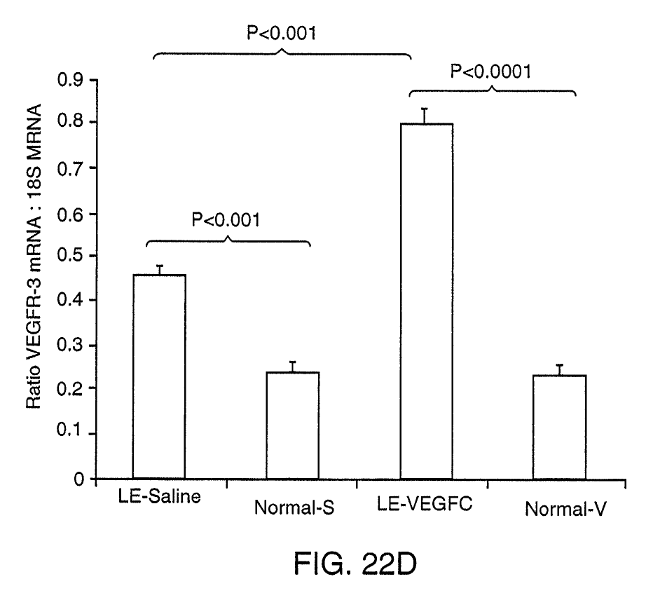

VEGFR-3 Expression in Rabbit Ear See FIGS. 22A-D.

We next investigated the VEGFR-3 expression by VEGF-C using semiquantitative RTPCR. First RT-PCR was performed on tissues from kidney, lymph node, lung and mesentery, which are known to express VEGFR-3 in other animals, to verify primer specificity. Next, quantitative-competitive RT-PCR was performed. Co-amplification of VEGFR-3 and 18S mRNA resulted in two distinct bands. Densitomety of VEGFR-3 RT-PCR product/18S RT-PCR product reveals a nearly 1.7 fold induction of VEGFR-3 mRNA levels by VEGF-C compared to control, (p<0.001). These data suggest that VEGFR-3 mRNA levels were observed to be strongly up-regulated in phVEGF-C transferred ears.

The rabbit VEGFR-3 amino acid sequence is shown in FIG. 22A.

### Example 10

### Mouse Tail Model of Lymphedema

Male nude (nu/nu) mice(Harlan) of 12 weeks of age were used. Anesthesia was achieved with intraperitoneal injections of 2% avertin 0.4 ml. The proximal portion of the tail was prepared by shaving and the operative site at the base of the tail was cleansed with 70% ethanol and povidone/iodine. Circumferential skin incisions were made with dissecting scissors around the base of the tail and removed skin and subcutaneous tissues to sever the superficial lymphatic network, without damaging arteries and veins, except for the central portion(1 to 2-mm width) of the dorsal skin. Both sides of the skin edges were cauterized to maintain hemostasis and a 3- to 4-mm gap was established for secondary healing.

In both experimental groups, tails started to swell a few hours after surgery. The increasing thickness of tails, was measured at the point just distal to the skin bridge with a vernier caliper by both horizontal and vertical axis. The tail thickness was defined as the average of the vertical and horizontal thickness. The thickness was measured before surgery and every week until 6 weeks.

See also Slavin S A, Van den Abbeele A D, Losken A, Swartz M A, Jain R K. Return of lymphatic function after flap transfer for acute lymphedema. Ann Surg 1999;229:421-427.

### Example 11

### Gene Transfer Protocol in Mouse Tail Model

Total 48 mice were divided into three groups in a blinded, randomized fashion before operation. No-operation group was used as negative control, sham-operation group was undertaken operation with no treatment, saline-injected group received operation and injected with saline and VEGF-C group received operation with gene transfer of VEGF-C. In the VEGF-C group, 100 μg of phVEGF-C in 100 μ volume was injected at and around the skin bridge using a 27-gauge needle intradermally and subcutaneously at post-operative days 1, 6 and 11, respectively. In the saline group, the same volume of saline was injected in an identical fashion.

Immunohistochemistry and Morphometric Analysis

The mice were sacrificed at various time points after gene injection. Skin from the site of injection was fixed in 4% paraformaldehyde and embedded in paraffin, and 5-μm sections were stained using monoclonal antibodies against VEGFR-3 or polyclonal antibodies against the lymphatic marker LYVE-1, a receptor for hyaluronan and a homologue to the CD44 glycoprotein. The tyramide signal amplification(TSA) kit(NEN Life Sciences) was used to enhance staining. Negative controls were done by replacing the primary antibodies with IgG of the same species from primary antibody was produced. The results were viewed with an Olympus microscope and photographed. For quantification, the vessels in the sections were counted under ×200 magnification. Eight visual fields were randomly selected and quantified in each mice (n=5, each group).

See also Banerji S, Ni J, Wang S X, Clasper S, Su J, Tammi R, Jones M, Jackson D G. LYVE-1, a new homologue of the CD44 glycoprotein, is a lymph-specific receptor for hyaluronan. J Cell Biol 1999;144:789-801.

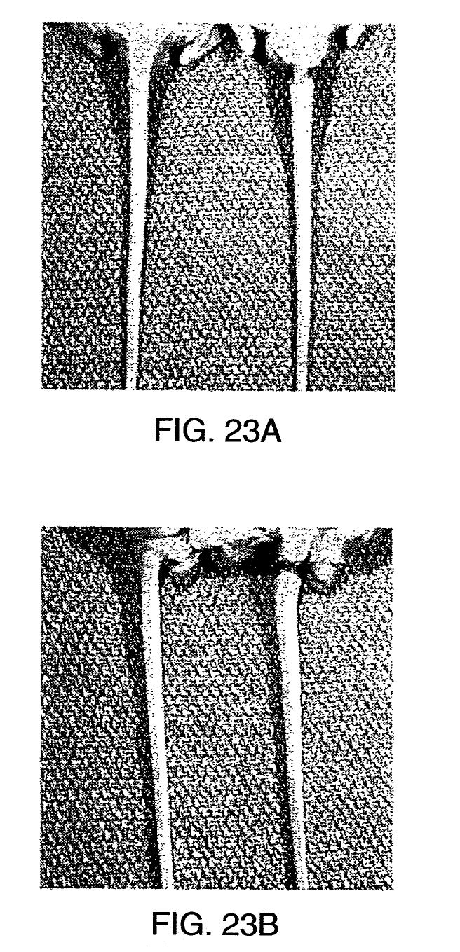

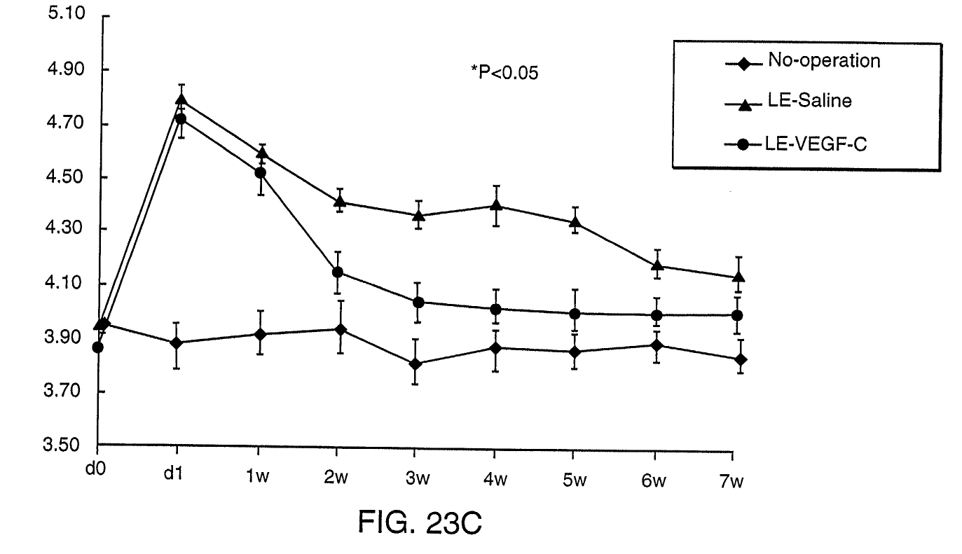

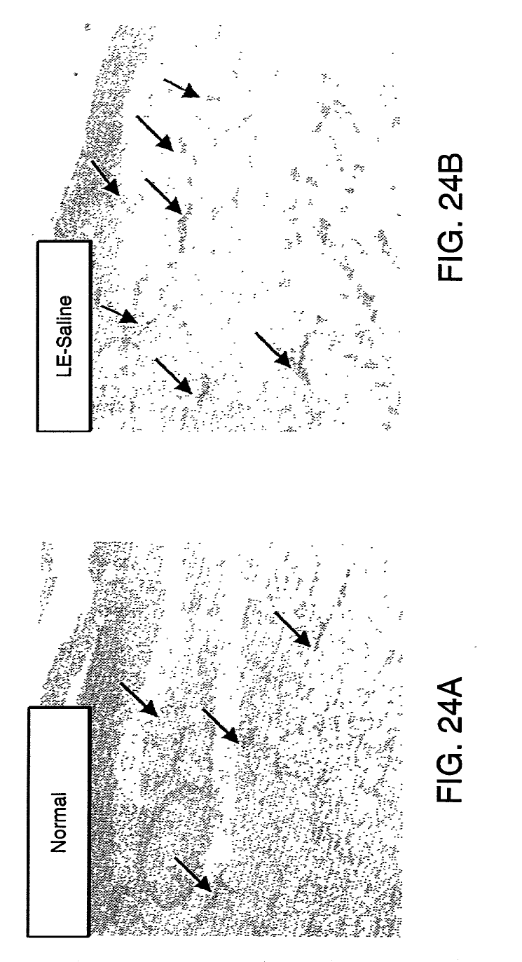

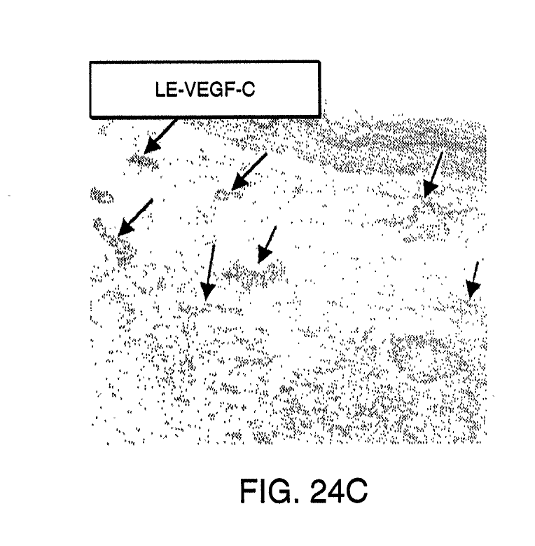

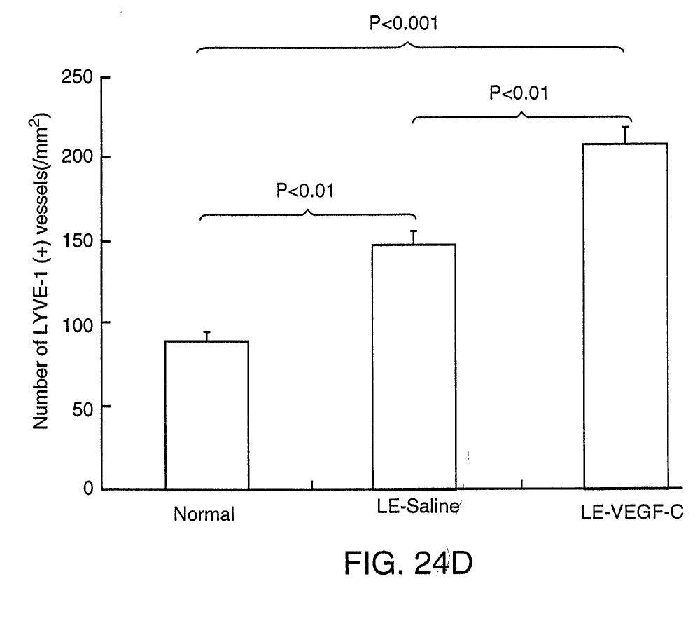

It was found that VEGF-C gene transfer improved lymphedema in a mouse tail model of lymphedema. See FIGS. 23A-C.

To determine whether the effect of phVEGF-C could be reproduced on another lymphedema model, we modified previously published mouse tail model of lymphedema. All the operated tail developed significant post-operative lymphedema from post-op day 1 which sustained over the 6 week follow-up. To assess the degree of lymphedema, we measured the thickness at proximal part of tail. The tail thickness measured by a caliper was significantly greater in the operated tail than the non operated tail at any time points from post-op 1 week to 6 weeks. In VEGF-C transferred tail, compared to the normal saline treated tail, the tail thickness was significantly lower at 3 and 4 weeks(P<0.05).

Gene Transfer of VEGF-C Promoted Lymphangiogenesis See FIGS. 29A-D.

The tail skins at the site of operation, from phVEGF-C, normal saline injected or non-operated group were processed for immunohistochemistry and stained for LYVE-1. As can be seen from the Figure, phVEGF-C transferred samples showed significantly increased density of LYVE-1-positive lymphatic vessels compared to either normal saline treated or normal tail samples(P<0.01).

Statistical Analysis

All results were expressed as the mean ± standard error of the mean (mean±SEM). Statistical analysis was performed by an unpaired Student's t-test for comparisons between two groups and ANOVA followed by Scheffe's procedure for more than two groups. P-values<0.05 were considered to denote statistical significance.

The following references are referred to by number in Examples 5-11, above. The disclosures of each reference are specifically incorporated by reference.

1. Browse N L. The diagnosis and management of primary lymphedema. J Vasc Surg 1986;3:181-184.

2. Anon. Lymphatic filariasis-tropical medicine's origin will not go away. Lancet 1987;1:1409

3. Szuba A, Rockson S G. Lymphedema: classification, diagnosis and therapy. Vascular Medicine 1998;3:145-156.

4. Ko DS, Lemer R, Klose G, Cosimi AB, Effective treatment of lymphedema of the extremities. Arch Surg 1998;134:452-458.

5. Slavin S A, Upton J, Kaplan W D, van den Abbeele A D. An investigation of lymphatic function following tissue transfer. Plast Reconstr Surg 1997;99:730-741.

6. Drinker C K, Field M E, Homans J. The experimental production of edema and elephantiasis as a result of lymphatic obstruction. Am J Physiol 1934;108:509.

7. Clodius L, Wirth W A. A new experimental model of chronic lymphedema of the extremities. Chir Plast(Berl)1974;2:115

8. Casley-Smith J R, Clodius L, Piller N B, Foldi M. A model of lymphema in the rabbits ear. The effect of benzopyrones. Chir Plast 1977;4:5-14.

9. Lee-Donaldson L, Witte M H, Bernas C L, Witte D, Stea W B. Refinement of a rodent model of peripheral lymphedema. Lymphology 1999;32:111-117.

10. Leak L V, Jones M. Lymphangiogenesis in vivo: formation of lymphatic capillary-like channels from confluent monolayers of lymphatic endothelial cells. In Vitro Cell Dev Biol 1994;30A:512-518.

11. O h S J, Jeltsch M, Birkenhager R, McCarthy J, Weich H, Christ B, Alitalo K, Wilting J. VEGF and VEGF-C: specific induction of angiogenesis and lymphangiogenesis in the differentiated avian chorioallantoic membrane. Dev Biol 1997;188:96-109.

12. Aprelikova O, Pajusola K, Partanen J, Armstrong E, Alitalo R, Bailey S K, McMaho J, Wasmuth J, Huebner K, Alital K. FLT-4, a novel class III receptor tyrosine kinase in chromosome 5q33-qter. Cancer Res 1992;52:746-748.

13. Galland F, Karamysheva A, Mattei M G, Rosnet O, Marchetto S, Birnbaum D. Chromosomal location of FLT-4, a novel receptor tyrosine kinase gene . Genomics 1992;13:475-478.

14. Lee J, Gray A, Yuan J, Luoh S M, Avraham H, Wood W I. Vascular endothelial growth factor-related protein: a ligand and specific activator of the tyrosine kinase receptor Flt-4. Proc Nat Acad Sci 1996;93:1988-1992.

15. Joukov V, Pajusola K, Kaipainen A, Chiloy D, Lahtinen I, Kukk E, Saksela O, Kalkkinen N, Alitalo K. A novel vascular endothelial growth factor, VEGF-C is a ligand for the Flt4(VEGFR-3) and KDR(VEGFR-2) receptor tyrosine kinases. EMBO J 1996;15:290-298.

16. Kaipainen A, Korhonen J, Mustonen T, van Hinsbergh V W M, Fang G H, Dumont D, Breitman M, Alitalo K. Expression of the fins-like tyrosine kinase 4 gene becomes restricted to lymphatic endothelium during development. Proc Nat Acad Sci USA 1995;92:3566-3570.

17. Kukk E, Lymboussaki A, Taira S, Kaipainen A, Jeltsch M, Joukov V, Alitalo K. VEGFC receptor binding and pattern of expression with VEGFR-3 suggests a role in lymphatic vascular development. Development 1996;122:3829-3837.

18. Jeltsch M et al. Hyperplasia of lymphatic vessels in VEGF-C transgenic mice. Science 1997;276:1423-1425.

19. Skobe M, Hawighorst T, Jackson DG, Prevo R, Janes L, Velasco P, Riccardi L, Alitalo K, Claffey K, Detmar M. Induction of tumor lymphangiogenesis by VEGF-C promotes breast cancer metastasis. Nature Med 2001;7:192-198.

20. Mandriota S J, Jussila L, Jeltsch M, Compagni A, Baetens D, Prevo R, Banerji S, Huarte J, Montesano R, Jackson D G, Orci L, Alitalo K, Christofori G, Pepper M S. Vascular endothelial growth facotor-C-mediated lymphangiogenesis promotes tumor metastasis. EMBO J 2001;20:672-682.

21. Ferrell R E, Levinson K L, Esman J H, Kirmak M A, Lawrence E C, Barmada M M, Finegold D N. Hereditary lymphedema: evidence for linkage and genetic heterogeneity. Hum Mol Genet 1998;7:2073-3078.

22. Karkkainen M J, Ferrell R E, Lawrence E C, Kimak M A, Levison K L, McTigue M A, Alitalo K, Finegold D N. Missense mutations interfere with VEGFR-3 signalling in primary lymphoedema. Nature genetics 2000;25:153-159.

23. Makinen T, Jussila L, Veikkola T, Karpanen T, Kettunen M I, Pulkkanen K J, Kauppinen R, Jackson D, Kubo H, Nishikawa S, Yla-Herttuala S, Alitalo K. Inhibition of lymphangiogenesis with resulting lymphedema in transgenic mice expressing soluble VEGF receptor-3. Nature Med 2001;7:199-205.

24. Piller N B, Clodius L. Lymphoedema of the rabbit ear following partial and complete lymphatic blockade; its effects on fibrotic development, enzymatic types and their activity levels. Br J Exp Path 1978;59:319-326.

25. Fu K, Izquierdo R, Vandevender D, Warpeha R L, Fareed J. Transplantation of lymph node fragmens I a rabbit ear lymphedema model: a new method for restoring the lymphatic pathway. Plast Reconstr Surg 1998;101:134-141.

26. Hung J C, Wiseman G A, Wahner H W, Mullan B P, Taggart T R, Dunn W L. Filtered technetium-99m-sulfur colloid evaluated for lymphoscintigraphy. J Nucl Med 1995;36:1895-1901.

27. Joukov V, Sorsa T, Kumar V, Jeltsch M, Claesson-Welsh L, Cao Y, Saksela O, Kalkkien N, Alitalo K. Proteolytic processing regulates receptor specificity and activity of VEGFC. EMBO J 1997;16:3898-3911.

28. Rivard A, Fabre J E, Silver M, Chen D, Murohara T, Kearney M, Magner M, Asahara T, Isner J M. Age-dependent impairment of angiogenesis. Circulation. 1999;99:111-120.

29. Lymboussaki A, Olofsson B, Eriksson U, Alitalo K. Vascular endothelial growth factor(VEGF) and VEGF-C show overlapping binding sites in embryonic endothelia and distinct sites in differentiated adult endothelia. Circ Res 1999;85:992-999.

30. Baumgartner I, Pieczek A, Manor O, Blair R, Kearney M, Walsh K, Isner JM. Constitutive expression of phVEGF165 following intramuscular gene transfer promotes collateral vessel development in patients with critical limb ischemia. Circulation 1998;97:1114-1123.

31. Casley-Smith J R, Foldi M, Ryan T J et al. Lymphedema, summary of the 10th internaltional congress of lymhology: working group discussion and Recommendations, Adelaide, Austria, August 10-17, 1985. Lymphology 1985;18:175-180.

32. Weissleder H, Weissleder R. Lymphedema; Evaluation of qualitative and quantitative lymphoscintigraphy in 238 patients. Radiology 1988;167:729-735.

33. Howarth D M. Increased lymphoscintigraphic flow pattern in the lower extremity under evaluation for lymphedema. Mayo Clin Proc 1997;72:423-429.

34. Anthony J, Foster R, Price D M, Mahdavian M, Inoue Y. Lymphatic regeneration following microvascular limb replantation; a qualitative and quantitative animal study. J Reconstr Microsurg 1997; 13:327-330.

See also the following references, the disclosures of which are incorporated herein by reference.

35. Pepper M S, Mandriota S J, Jeltsch M, Kumar V, Alitalo K. Vascular endothelial growth factor(VEGF)-C synergizes with basic fibroblast growth factor and VEGF in the induction of angiogenesis in vitro and alters endothelial cell extracellular proteolytic activity. J Cell Physiol 1998;177:439-52.

36. Finnerty H, Kelleher K, Morris G E, Bean K, Merberg DM, Kriz R, Morris J C, Sookdeo H, Turner K J, Wood C R. Molecular cloning of murine FLT and FLT4. Oncogene 1993;8:22932298.

37. Galland F, Karamysheva A, Oebusque M J, Borg J P, Rottapel R, Dubreuil P, Rosnet O, Birnbaum D. The FLT4 gene encodes a transmembrane tyrosine kinase related to the vascular endothelial growth factor receptor. Oncogene 1993;8:1233-1240.

38. Schratzberger P, Walter D H, Rittig K, Bahlmann F H, Pola R, Curry C, Silver M, Krainin J G, Weinberg D H, Ropper A H, Isner J M. Reversal of experimental diabetic neuropathy by VEGF gene transfer. J Clin Invest 2001;107:1083-1092.

39. Witzenbichler B, Asahara T, Murohara T, Silver M, Spyridopoulos I, Magner M, Principe N, Kearney M, Hu J S, Isner J M. Vascular endothelial growth factor-C(VEGF-C/VEGF2) promotes angiogenesis in the setting of tissue ischemia. Am J Pathol 1998;153:381-394.

40. Enholm B, Karpanen T, Jeltsch M, Kubo H, Stenback F, Prevo R, Jackson D G, YlaHerttuala S, Alitalo K. Adenoviral expression of vascular endothelial growth factor-c induces lymphangiogenesis in the skin. Circ Res 2001;88:623-29.

The invention has been described with reference to preferred embodiments thereof. However, it will be appreciated that those skilled in the art, upon consideration of this disclosure, may make modifications and improvements within the spirit and scope of the invention.

What is claimed is:

1. A method for inducing formation of new lymphatic vessels in a mammal, wherein the method comprises administering to the mammal an effective amount of vascular endothelial growth factor (VEGF) or an effective fragment thereof sufficient to form the new vessels in the mammal.

2. The method of claim 1, wherein the amount of the VEGF administered to the mammal is sufficient to decrease ear volume by at least about 10% as determined by a standard rabbit ear assay.

3. The method of claim 1, wherein the amount of the VEGF administered to the mammal is sufficient to increase the number of lymphatic vessels by at least about 10% as determined by a standard lymphoscintigraphy assay.

4. The method of claims 1-3, wherein the administered VEGF comprises or consists of VEGF-2; or an effective fragment thereof.

5. The method of claims 1-4, wherein the amount of the VEGF administered to the mammal is sufficient to increase growth of new lymphatic vessels following lymphedema.

6 The method of claim 5, wherein the increase in new lymphatic vessel grow is at least about 10% as determined by standard lymphoscintigraphy.

7. The method of claim 1, wherein the mammal has, is suspected of having, or will have lymphedema or a medical condition associated with same such as lymphangietasia, lymphangioma, and lymphangiosarcoma.

8. The method of claim 7, wherein the lymphedema is primary or secondary lymphedema.

9. The method of claims 1-8, wherein the VEGF is co-administered with at least one angiogenic protein.

10. A method for preventing or reducing the severity of lymphatic vessel damage in a mammal, wherein the method comprises administering to the mammal an effective amount of vascular endothelian growth factor (VEGF); and exposing the mammal to conditions conducive to damaging the lymphatic vessels, the amount of VEGF being sufficient to prevent or reduce the severity of the vessel damage in the mammal.

11. The method of claim 10, wherein the conditions conducive to the lymphatic vessel damage are an invasive manipulation, disease, genetic predisposition, congential (onset less than about two years after birth), lymphedema precox, lymphedema tarda, or trauma.

12. The method of claim 11, wherein the invasive manipulation is surgery such as ilio-femoral bypass, regional lymph node dissection including axillary (post-mastectomy lymphedema), pelvic and para-aortic (leg and groin lymphedema), and neck (head and neck lymphedema).

13. The method of claim 11, wherein the disease is a neoplastic disease, rheumatoid arthritis, filariasis or recurrent infection such as erysipelas.

14. The method of claim 13, wherein the neoplastic disease is hodgkin lymphoma, metastatic cancer, or a cancer of the prostate or breast, cervical cancer or melanoma.

15. The method of claim 11, wherein the trauma is associated with a medial aspect of a thigh.

16. The method of claim 11, wherein the genetic predisposition is familial autosomal dominant.

17. The method of claim 16, wherein the predisposition is Nonne-Milroy disease.

18. The method of claim 11, wherein the genetic predisposition is familial and non-dominant.

19. The method of claim 11, wherein the congential lymphatic vessel damage is sporadic.

20. The method of claim 11, wherein the lymphedema precox (onset between about 2 and 35 years of age) is familial, autosomal recessive such as Meige disease.

21. The method of claim 11, wherein the lymphedema precox is sporadic.

22. The method of claim 8, wherein the primary lymphedema is associated with one or more of a distal obliteration, proximal obliteration, or hyperplasia.

23. The method of claims 7-22, wherein the VEGF is administered to the mammal at least about 12 hours before exposing the mammal to the conditions conducive to damaging the lymphatic vessels.

24. The method of claim 23, wherein the VEGF is administered to the mammal between from about 1 to 10 days before exposing the mammal to the conditions conducive to damaging the vessels.

25. The method of claims 22-24, wherein the method further comprises administering the VEGF to the mammal following the exposure to the conditions conducive to damaging the vessels.

26. A method for treating lymphedema in a mammal in need of such treatment, wherein the method comprises administering to the mammal an effective amount of vascular endothelial growth factor (VEGF) or an effective fragment thereof sufficient to form the new vessels in the mammal.

27. The method of claim 26, wherein the VEGF comprises or consists of VEGF-2; or an effective fragment thereof.

28. The method of claims 25-27 further comprising co-administering at least one angiogenic protein.

29. The methods of claims 1-28, wherein the mammal is a rabbit, rodent or a primate.

30. The method of claim 29, wherein the primate is a human patient.

31. A pharmaceutical product for inducing growth of new lymphatic vessels in a mammal, wherein the product comprises vascular endothelian factor 2 (VEGF-2) or an effective fragment thereof.

32. The pharmaceutical product of claim 31 in which the VEGF-2 is formulated to be physiologically acceptable to a mammal.

33. The pharmaceutical product of claim 32, wherein the product is sterile and comprises VEGF-2 protein or nucleic acid encoding the protein.

34. A kit for the treating lymphedema in a human patient, wherein the kit comprises VEGF-2 protein, nucleic acid encoding VEGF, or an effective fragment thereof, the kit optionally further comprising a pharmacologically acceptable carrier solution, means for delivering the VEGF-2 protein or nucleic acid and directions for using the kit.

35. The kit of claim 34, wherein the means for delivering the VEGF-2 protein or nucleic acid is a stent, catheter or syringe.

36. A test system for identifying compounds that reduce lymphedema, the system comprising:
a) a mammal characterized by having a surgically manipulated appendage, the manipulation being sufficient to expose a neurovascular bundle (NVB) in the ear and to provide a substrate for detecting neolymphatic growth,
b) a candidate compound for reducing lymphedema in the mammal; and
c) at least one implementation for detecting an increase or decrease in appendage thickness following contact of the candidate compound with the NVB.

37. The test system of claim 36, wherein the mammal is a rabbit.

38. The test system of claims 36-37, wherein the candidate compound comprises vascular endothelian factor (VEGF); or an effective fragment thereof.

39. The test system of claim 38, wherein the VEGF is VEGF-2 or an effective fragment thereof.

40. The rabbit VEGFR-3 cDNA sequence shown in FIG. 21.

41. The rabbit VEGFR-3 amino acid sequence shown in FIG. 22A.
